(19) United States
(12) Patent Application Publication
(10) Pub. No.: US 2009/0214590 A1
(43) Pub. Date: Aug. 27, 2009

(54) Virus Vaccines Comprising Envelope-Bound Immunomodulatory Proteins and Methods of Use Thereof

(75) Inventors: Roy Sundick, Farmington Hills, MI (US); Paul Christopher Roberts, Blacksburg, VA (US); Yufang Yang, Windsor, MI (US); Andrew Scott Herbert, Blacksburg, VA (US)

Correspondence Address: DARBY & DARBY P.C., P.O. BOX 770, Church Street Station, New York, NY, 10008-0770, US

(73) Assignee: Wayne State University, Detroit, MI (US)

(21) Appl. No.: 11/988,358

(22) PCT Filed: Jul. 10, 2006
(86) PCT No.: PCT/US06/26927

§ 371 Date: Jan. 4, 2008

Related U.S. Application Data
(60) Provisional application No. 60/697,777, filed on Jul. 8, 2005.

(51) Int. Cl. A61K 39/12; C12N 7/01

(52) U.S. Cl. 424/205.1; 435/235.1

(57) ABSTRACT

The present invention provides novel virus vaccines with augmented, e.g., enhanced and/or extended immunogenicity. The virus vaccines of the invention comprise an envelope-bound immunomodulatory protein, e.g., a cytokine, chemokine or costimulatory molecule. The immunomodulatory protein serves as an adjuvant to augment, e.g., enhance or extend the immunogenicity of the virus vaccine, thereby augmenting, e.g., enhancing or extending immune response to the virus when administered to a subject.

### CROSS-REFERENCE TO RELATED APPLICATIONS

This application is a national phase of International Application No. PCT/US2006/26927, filed Jul. 10, 2006, which claims the benefit of U.S. Provisional Application No. 60/697,777, filed Jul. 8, 2005. International Application No. PCT/US2006/26927 published in English on Jan. 18, 2007 under Publication No. WO 2007/008918. These applications are hereby incorporated by reference in their entirety.

### BACKGROUND OF THE INVENTION

The successful elimination of pathogens following prophylactic or therapeutic immunization depends to a large extent on the ability of the host's immune system to become activated in response to immunization and to mount an effective response, preferably with minimal injury to healthy tissue.

Among the most established ways for increasing the immunogenicity of antigens is the use of immunoenhancing agents, or “adjuvants”. Adjuvants accelerate, prolong, and/or enhance an antigen-specific immune response as well as provide the selective induction of the appropriate type of response. (Ramachandra L, et al., Immunol. Rev. 1999; 168:217-239; Singh M, et al., Nat. Biotechnol. 1999; 17:1075-1081). In the absence of an adjuvant, reduced or no immune response may occur, or worse the host may become tolerized to the antigen.

Adjuvants can be found in a group of structurally heterogeneous compounds (Gupta et al., Vaccine 1993; 11:293-306). Classically recognized examples of adjuvants include oil emulsions (e.g., Freund's adjuvant), saponins, aluminium or calcium salts (e.g., alum), non-ionic block polymer surfactants, lipopolysaccharides (LPS), mycobacteria, and many others. Theoretically, each molecule or substance that is able to favor or amplify a particular situation in the cascade of immunological events, ultimately leading to a more pronounced immunological response, can be defined as an adjuvant.

In principle, through the use of adjuvants in vaccine formulations, one can (1) direct and optimize immune responses that are appropriate or desirable for the vaccine; (2) enable mucosal delivery of vaccines, i.e., administration that results in contact of the vaccine with a mucosal surface such as buccal, gastric, nasal or lung epithelium and the associated lymphoid tissue; (3) promote cell-mediated immune responses; (4) enhance the immunogenicity of weaker immunogens, such as highly purified or recombinant antigens; (5) reduce the amount of antigen or the frequency of immunization required to provide protective immunity; and (6) improve the efficacy of vaccines in individuals with reduced or weakened immune responses, such as newborns, the aged, and immunocompromised vaccine recipients.

Numerous studies report the adjuvant properties of cytokines (reviewed by Schijns, V. E., Vet Immunol Immunopathol, 2002; 87(3-4):195-8; Calarota S A, Weiner D B., Immunol Rev. 2004 June; 199:84-99; O'Hagan, D. T., Curr Drug Targets Infect Disord, 2001, 1(3): 273-86), chemokines (Flanagan, K., et al., Vaccine, 2004; 22(21-22): p. 2894-903; Toka, F. N., C. D. Pack, and B. T. Rouse, Immunol Rev, 2004; 199:100-12), and costimulatory molecules, including CD80 and CD86 (Cimino A M, Palaniswami P, Kim A C, Selvaraj P., Immunol Res. 2004; 29(1-3):231-40; Calarota S A, Weiner D B., Immunol Rev. 2004 June; 199:84-99 (Review)) in vaccine formulations. Several studies demonstrate the efficacy of cytokines in boosting immune responses to the influenza virus (Faulkner, L., et al., Int. Immunol. 2001; 13(6):713-21; Moran, T. M., et al., J Infect Dis. 1999; 180(3):579-85). Faulkner et al. (2001) fused an immunodominant T cell epitope of HA with IL-2 and observed enhanced T cell activation compared to controls stimulated with HA and unlinked IL-2. Moran et al. (1999) immunized mice with inactivated influenza co-administered with IL12 and antibodies to IL4. This regimen switched responses from TH2 to TH1 and induced enhanced protection to challenge with heterosubtypic virus. Subunit influenza vaccines composed of liposome-encapsulated HA/NA and IL-2 or GM-CSF were successfully used in mice to stimulate both TH1 and TH2 responses (Babai, I., et al., Vaccine 1999; 17(9-10): 1223-38; Babai, I., et al., Vaccine 1999; 17(9-10):1239-50).

Previous studies in animals (reviewed by Naylor, P. H. and J. W. Hadden, Int. Immunopharmacol. 2003; 3(8):1205-15), including chickens (Hulse, D. J. and C. H. Romero, Vaccine 2004; 22(9-10): 249-59; Hu, W., et al., Current Progress on Avian Immunology Research, ed. K. A. Schat. 2001, American Association Avian Pathologists: Kennett Square. 269-274), have demonstrated the adjuvanticity of IL-2 administered with several viral vaccines. For example, IL-2 has been widely used as a vaccine adjuvant, in the form of protein, DNA vaccine (Scheerlinck, J. P., et al., Vaccine 2001; 19 (28-29):4053-60) and as a gene incorporated into viral and bacterial vectors (Bukreyev, A. and I. M. Belyakov, Expert Rev Vaccines 2002; 1(2): 233-45; Ghiasi, H., et al. J. Virol., 2002; 76(18): 9069-78). In general, IL-2 boosts both antibody and T cell mediated responses, including T cytotoxic responses.

For example, it has been shown that the administration of chicken IL2 adsorbed to beads coated with the glycoprotein B of Marek's disease virus increased both antibody and T cell proliferative responses to gB (Hu, W., et al., Current Progress on Avian Immunology Research, ed. K. A. Schat. 2001, American Association Avian Pathologists: Kennett Square. 269-274). IL-15, which shares the same tertiary structure with IL-2, has proven effective in several experimental vaccines (Umemura, M., et al., Infect. Immun. 2003; 71(10): 6045-8; Min, W., et al., Vet. Immunol. Immunopathol. 2002; 88(1-2):49-56; Min, W., et al., Vaccine 2001; 20(1-2): 267-74; Lillehoj, H. S., et al., Vet. Immunol. Immunopathol. 2001; 82(3-4):229-44). Both cytokines have been cloned in chickens (Lillehoj, H. S., et al. (2001) and Sundick, R. S, and C. Gill-Dixon, J. Immunol. 1997; 159(2):720-5). The biological properties of chicken IL-18 have recently been characterized (Gobel, T. W., et al., J. Immunol. 2003; 171(4):1809-15). Chicken IL-18, similar to its mammalian homolog, induces interferon gamma, upregulates MHC class II expression and stimulates the proliferation of CD4 T cells. IL-8, a potent proinflammatory chemokine, acts on multiple cells, including neutrophils, lymphocytes, monocytes and endothelial cells (Min, W., et al., Vaccine 2001; 20(1-2):267-74 and Mukaida, N., Am. J. Physiol. Lung Cell. Mol. Physiol. 2003; 284(4):L566-77). Other cytokines have been cloned in chickens, including IL-1, interferon gamma, IL-4, G-CSF, IL-12 (Degen W G, van Daal N, van Zuilekom H I, Burnside J, Schijns V E., J. Immunol. 2004 Apr. 1; 172(7):4371-80) and GM-CSF (Avery S, Rothwell L, Degen W D J, Schijns V E, Young J, Kaufman J, Kaiser P., J Interferon Cytokine Research 2004; 24:600-614).

There is a need for improved vaccines with the ability to direct the immune response towards generation of potent neutralizing antibody responses and a potent cellular response in various animals, including humans and agriculturally important animals.

### SUMMARY OF THE INVENTION

The present invention takes advantage of the immunostimulatory properties of cytokines, chemokines and costimulatory molecules as a means to augment, e.g., enhance and/or extend, immune response to antigens and to produce novel vaccine formulations. The present invention provides novel methods and compositions for augmenting the immunogenicity of a virus vaccine by tethering an immunomodulatory protein, e.g., a cytokine, chemokine or costimulatory molecule to the viral envelope, to enhance and/or extend immune response to the virus in a subject.

In one aspect, the present invention is directed to a composition comprising an enveloped virus expressing an envelope-bound, immunomodulatory protein linked to a viral envelope protein, or fragment thereof. In a preferred embodiment, the virus is inactivated. In one embodiment, the immunomodulatory protein is linked to the amino-terminal domain of the viral envelope protein, or fragment thereof. In another embodiment, the amino-terminal domain comprises the transmembrane domain and the cytoplasmic domain of a viral envelope protein. In a specific embodiment, the viral envelope protein is selected from the group consisting of neuraminidase (NA) and hemagglutinin (HA), hemagglutinin-neuraminidase (HN) or hemagglutinin-esterase-fusion (HEF) glycoproteins from viruses belonging to the Orthomyxoviridae family. Other specific embodiments include viral envelope glycoproteins, including but not limited to E2/E1, E, gp62 (Togaviridae); EFPgp64 (Baculoviridae); HN, F, H or G (Paramyxoviridae), G (Rhabdoviridae); gp41, gp37, p15E, gp36, gp22, gp30, gp48 (Retroviridae); E, HE, S, M (Coronoviridae); G1/G2 (Bunyaviridae); gG, gE, gI, gD, gJ, gK, gC, gB, gH, gM, gL, gp85/BXLF2, gp25/BKRF2, gp110 BALF4, gp84/113/BBRF3, gp15/BLRF1 and gp 350/220 (Herpesviridae); A33R, A34R, A36R, A56R, B5R, H3L, 15L and A27L (Poxyiridae); GP (Filoviridae) G1, gPr90, gp51, gp52, gp55, gp70, gp120 and gp41, and E1 and E2.

The immunomodulatory proteins used in the invention include cytokines, chemokines or costimulatory molecules or fragments thereof. In one embodiment, the cytokine is selected from the group consisting of IL-1, IL-2, IL-4, IL-5, IL-6, IL-10, IL-12, IL-15, IL-18, GM-CSF, and interferon gamma. In another embodiment, the chemokine is selected from the group consisting of IL-8, SDF-1α, MCP1, MCP2, MCP3 and MCP4 or MCP5, RANTES, MIP-5, MIP-3, eotaxin, MIP-1α, MIP-1β, CMDC, TARC, LARC, and SLC. In another embodiment the costimulatory molecule is selected from the group consisting of CD80, CD86, ICAM-1, LFA-3, C3d, CD40L and Flt3L.

In one embodiment, the immunomodulatory protein is derived from the animal to which the composition is to be administered, e.g., an animal selected from the group consisting of a chicken, duck, goose, turkey, rodent, e.g., mouse, horse, cow, sheep, pig, monkey, dog, and cat. In a preferred embodiment, the immunomodulatory protein is a human immunomodulatory protein.

The virus used in the methods of the invention may be any enveloped virus. For example, a virus used in the invention may belong to the family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Bunyaviridae and Baculoviridae. In a preferred embodiment, the virus is selected from the group consisting of human influenza viruses, avian influenza viruses, parainfluenza viruses, respiratory syncitial virus (RSV), Hepatitis B, Hepatitis C, human immunodeficiency virus (HIV), human T-cell leukemia virus (HTLV-1/2), feline leukemia virus (FeLV), avian sarcoma virus, Herpesvirus, varicella-zoster virus (VZV), cytomegalovirus (CMV), lymphocytic choriomeningitis virus (LCMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

In another aspect, the invention provides methods for producing an enveloped virus expressing an envelope-bound, immunomodulatory protein, the method comprising a) transforming a host cell with an expression vector encoding an immunomodulatory protein fused to a viral envelope protein, or a fragment thereof; and b) infecting the cell with an enveloped virus, thereby producing an enveloped virus expressing an envelope-bound, immunomodulatory protein. In one embodiment, the host cell is an MDCK cell or other cell line permissive for growth of the respective virus. In another embodiment, the method further comprises inactivating the virus.

The present invention also provides methods for inducing an immune response in an animal comprising administering to the animal an effective amount of a composition comprising an inactive virus expressing an envelope-bound immunomodulatory protein, e.g., a cytokine, chemokine or costimulatory molecule, wherein the immune response induced by (or in) the animal is more robust, e.g., enhanced and/or extended, as compared to the immune response that could have been induced in an animal by the virus without the envelope-bound immunomodulatory protein. The immune response induced by the inactive virus may be a humoral immune response or a cellular immune response, e.g., a cytotoxic T cell and/or T helper cell mediated immune response. In another embodiment, the immune response is an innate response that directs the humoral and/or cellular responses.

Another aspect of the invention provides for methods for treating or preventing a viral infection in an animal comprising administering to the animal an inactive, enveloped virus expressing an envelope-bound immunomodulatory protein.

In one embodiment, the viral infection is caused by a virus belonging to a family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Baculoviridae and Bunyaviridae. In a preferred embodiment, the virus is selected from the group consisting of influenza viruses, e.g., human influenza viruses and avian influenza viruses, parainfluenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpesvirus, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

In a specific embodiment, the animal is selected from the group consisting of a chicken, duck, goose, turkey, rodent, e.g., mouse, horse, cow, sheep, pig, monkey, dog, and cat. In a preferred embodiment, the animal is a human.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 depicts immunofluorescence and phase contrast visualization of filamentous influenza A virus budding from the surface of infected MDCK cells. At 9 h.p.i, MDCK cell infected with A/Udorn/72 (m.o.i.=3) were sequentially incubated at 4° C. with anti-Udorn antisera and TRITC-goat anti-guinea pig Ig. The cells were fixed in 3% paraformaldehyde, permeabilized with 0.2% Triton X-100 and further incubated with anti-vimentin and FITC-conjugated rabbit anti-mouse Ig.

FIG. 2 is a graph depicting real-time RT-PCR of NA-chIL-2 mRNA levels in MDCK subclones transfected with the plasmid pcDNA3.1 encoding the fusion protein NA-chIL-2. Levels are expressed relative to vector, control MDCK cells.

FIG. 3 is a graph depicting the results of a bioassay for chicken IL-2 expressed in transfected MDCK cells. Clone 15 significantly (p<0.01) stimulated T blasts compared to control MDCK cells illustrating that NA-chIL2 expressed at the surface of MDCK cells is biologically active.

FIG. 4 depicts immunofluorescence micrographs of MDCK/NA˜chIL2 subclone 15 cells. MDCK/NA˜chIL2 (subclone 15) cells (a, b, d and e) or MDCK vector only control cells (c, f) were infected with or without A/Udorn/72 virus for 8 hours, fixed in 3% paraformaldehyde and sequentially incubated with monoclonal anti-chIL2 antibody (clone G) and AlexaFluor488˜conjugated goat anti-mouse Ig (a, b, c). In b, cells were additionally stained with rabbit anti-chIL2 antiserum and AlexaFluor594; note yellow colocalization for both antibodies. In virus infected cells (lower panel) cells were sequentially incubated with goat anti-hemagglutinin H3 antiserum, chicken anti-goat AlexaFluor594, anti-chIL2 mab G and goat anti-mouse AlexaFluor488. Note the incorporation of chIL2 into viral filaments budding from the cell surface (yellow colocalization with H3 antigen) in d and e, but not f.

FIG. 5 is a schematic of pcDNA3.1-based chicken cytokine/chemokine fusion constructs.

FIG. 6 is a graph illustrating bioactivity of UV-inactivated-A/Udorn virus particles. Virus harvested from wildtype (wild virus, black columns) or chIL2-expressing MDCK cells (virus-NAIL-2, white columns) was tested for IL-2 bioactivity, using mitogen-activated chicken T cell blasts as indicators. Recombinant soluble IL-2 was used as a positive control. Aliquots of virus treated with UV were found to be noninfective in cultures of MDCK cells.

FIG. 7 is a graph illustrating bioactivity of heat-inactivated-A/Udorn virus particles (56° C. for 20 minutes). Virus harvested from wildtype (wild virus, black columns) or chIL2-expressing MDCK cells (virus-NAIL-2, white columns) was heat-inactivated and tested for IL-2 bioactivity, using mitogen-activated chicken T cell blasts as indicators. Recombinant soluble IL-2 was used as a positive control. Aliquots of virus treated with heat were found to be noninfective in cultures of MDCK cells.

FIG. 8 illustrates results of examination of subclones of MDCK cell lines stably and constitutively expressing the fusion constructs NAmIL2 or mGM-CSF˜HA1513 for surface expression of mouse derived IL2 or GM-CSF using standard immunofluorescence staining techniques employing commercially available antibodies specific for mouse IL2 or mouse GM-CSF. (A) Positive staining specific for mouse IL2 using MDCK/NAmIL2 subclone 3 cells (rat anti-mouse IL2 antibody and Alexafluor 488˜chicken anti-rat Ig); (B) Positive staining specific for mouse GM-CSF using MDCK/mGMCSF.HA1513 subclone 4 cells (rat anti-mouse GM-CSF and Alexafluor 488˜chicken anti-rat Ig; and C) absence of staining in MDCK/pcDNA3.1 vector control cells (using rat anti-mouse IL2 antibody and Alexafluor 488 chicken anti-rat Ig).

FIG. 9A illustrates surface bioactivity of membrane-bound GM-CSF˜HA₁₅₁₃. Subclones (1-5) of MDCK cells expressing mouse GM-CSF˜HA₁₅₁₃ (white columns) were tested for GM-CSF bioactivity, using bone marrow cells as indicators. Recombinant mouse GM-CSF at 0.2 ng/ml and 0.04 ng/ml were used as positive controls. MDCK cells transfected with vector alone were used as a negative control.

FIG. 9B illustrates surface bioactivity of membrane-bound NA˜mIL2. MDCK cells transfected with NA˜mIL2 (white columns) were tested for mouse IL2 bioactivity, using CTTL2 cells as indicators. Recombinant mouse IL2 at 1 ng/ml and 0.2 and 0.04 ng/ml were used as positive controls (striped bars). MDCK cells transfected with chicken IL2 were used as a negative control (C-15, black bars).

FIG. 10 illustrates immunofluorescence staining of mouse GM-CSF˜HA¹⁵¹³ on MDCK cells and budding virions. MDCK cells transfected with mouse GMCSF≠HA1513 (c, d, e and t) or vector alone (a,b) were infected with A/Udorn virus and immunostained with anti-GMCSF or anti-HA antibodies and appropriate fluorophore conjugated secondary antibodies. Budding filamentous virions are indicated by arrows. Virions released into the supernatant were spun onto coverslips and stained with anti-GMCSF antisera. Virions from wildtype MDCK infected cells did not stain with anti-GMCSF antisera, but were positive when stained with anti-HA antibodies (data not shown).

FIG. 11 is a graph depicting results of an in vivo experiment utilizing inactivated influenza virus bearing IL2. Chicks were vaccinated, and boosted with wild-type virus or virus with membrane-bound IL2 in saline (PBS) or oil and tested for antiviral antibody by ELISA. The two groups injected with virus-chIL2 (cIL2), when combined, had significantly elevated mean antibody responses in comparison with the two groups of chicks vaccinated with wild-type virus.

FIG. 12 is a graph depicting results of a bioassay for chicken GM-CSF in MDCK cells expressing a chicken GM-CSF/HA construct. Influenza virus was grown on the MDCK cells expressing the chicken GM-CSF/HA construct and wild-type MDCK cells as controls. The virus was harvested and tested for GM-CSF bioactivity using bone marrow cells as indicators, bone marrow cells alone as a negative control and bone marrow plus GM-CSF as a positive control. Only the virus grown on MDCK cells expressing GM-CSF had significant bioactivity.

FIG. 13 is a graph depicting results of a bioassay for mouse IL2 in MDCK cells expressing a murine IL2/HA construct. Influenza virus was grown on the MDCK cells expressing the mIL2/HA construct. The virus was harvested and tested for IL2 bioactivity using CTTL2 cells as indicators, CTTL2 cells alone as a negative control and CTLL2+ConA activated supernatants (which contain soluble mouse IL2) as a positive control. Only the virus grown in MDCK cells transfected with mIL2/HA had significant bioactivity.

FIG. 14 is a graph depicting results of a bioassay for mouse IL4 in MDCK cells expressing a mouse IL4 construct. Influenza virus was grown on the MDCK cells expressing the mIL4/HA construct. The virus was harvested and tested for IL4 bioactivity using CT4.S cells as indicators, CT4.S cells alone as a negative control and CT4.S cells plus rmIL4 as a positive control. Only the virus grown in MDCK cells expressing mIL4/HA had significant bioactivity.

### DETAILED DESCRIPTION OF THE INVENTION

The present invention utilizes immunomodulatory proteins, e.g., cytokines, chemokines and costimulatory proteins, to produce novel vaccine formulations that augment, e.g., enhance and/or extend immune response when administered to a subject. The present invention provides a novel method for augmenting the immunogenicity of a virus vaccine by tethering an immunomodulatory protein, e.g., a cytokine, chemokine, or costimulatory protein, to the viral envelope, thereby enhancing immune response to the virus in a subject.

The present invention is based, at least in part, on the discovery of methods for producing viruses expressing an envelope-bound immunomodulatory protein, e.g., a cytokine, chemokine, or costimulatory protein, and vaccine compositions comprising these viruses. The immunomodulatory proteins bound to the envelope of a virus are active, i.e., they maintain their ability to enhance and/or extend an immune response when bound to the membrane of a virus, e.g., an inactivated virus, and thus are effective adjuvants. Thus, administration of viruses of the invention, e.g., inactivated virus vaccines, expressing envelope-bound, immunomodulatory proteins, to animal subjects results in an enhanced and/or extended immune response as compared to viruses that do not express an envelope-bound, immunomodulatory protein.

In one aspect of the invention, an expression vector is produced that encodes an immunomodulatory protein linked to a viral envelope protein, or a fragment thereof. The expression vector may be used to transfect cells, e.g., cells that allow productive virus replication, such as MDCK cells. These transfected cells may be selected for stable expression, e.g., by the use of a selective agent encoded by the plasmid (e.g., Geneticin) and cloned for maximal expression of immunomodulatory protein. The viral envelope protein (or fragment thereof) directs the immunomodulatory protein to the surface of the infected cell, where it is expressed on the cell membrane. In another embodiment, the viral envelope protein or fragment thereof directs the immunomodulatory protein to an intracellular membrane, from whence the virus will bud. When the cell is infected with an enveloped virus, e.g., an influenza virus, the virus buds from the cell surface or intracellular membrane expressing the immunomodulatory protein, e.g., cytokine, chemokine or costimulatory molecule, resulting in the an enveloped virus carrying the protein attached to the viral envelope. Therefore, the present invention is directed, at least in part, to methods for genetically modifying a virus-producing cell line so that it produces a membrane-bound variant of an immunomodulatory molecule, e.g., a cytokine or chemokine or costimulatory molecule. The present invention also includes expression vectors encoding envelope virus proteins, or fragments thereof, linked to an immunomodulatory protein.

In another embodiment, the present invention includes the use of the immunomodulatory molecules of the invention to produce vaccines comprising virus-like particles that incorporate the immunomodulatory molecules described herein. For example, expression of retroviral viral membrane proteins, e.g., gag-pol or gag alone with the viral env protein in producer cell lines (e.g., 293T cells or H9 cells), induces budding of virus-like particles. The virus-like particles are safe as vaccines since no viral nucleic acid is enclosed in the particles, and have been previously shown to be immunogenic in monkeys. Importantly, they have been shown to reduce viral load when monkeys are challenged with infectious virus (Wagner R, et al., Virology 1998 May 25; 245(1):65-74; Yao Q, et al., J. Immunol. 2004 Aug. 1; 173(3):1951-8; Kang C Y, et al., Biol. Chem. 1999 March; 380(3):353-64; and Singh D K, et al., J. Virol. 2005 March; 79(6):3419-28, the contents of which are included herein by reference). However, protection was not complete; virus replicated and persisted in the monkeys. The addition of membrane-bound immunomodulatory molecules, as described herein, to these particles, will enhance their protective efficacy. It will be understood that the virus-like particles of the invention are not limited to those derived from any particular virus.

Advantages of the invention, as compared to fusion proteins comprising immunomodulatory proteins that are not bound to the viral envelope, include, but are not limited to the following: 1) the immunomodulatory protein can serve as an adjuvant for all antigens associated with the virus, not just the one to which it is fused; 2) the immunomodulatory protein is produced in a eukaryotic cell so that it is glycosylated, folded properly and produced economically because there is no extra processing of the cytokine separate from that needed to purify virus; and 3) the viral-bound immunomodulatory protein may have a longer in vivo half-life than soluble cytokine fusion proteins.

As demonstrated in the Examples section, infra, a construct comprising the gene coding for mature chicken interleukin-2 (IL-2) linked to the gene fragment coding for the amino terminus of the neuraminidase (NA) gene of influenza virus, i.e., a gene segment that codes for the intracytoplasmic domain, the transmembrane domain and 19 amino acids of the extracellular domain of NA, has been produced. This construct was ligated into an expression plasmid and transfected into MDCK cells. A subclone of the transfected cells was isolated that expressed active, chimeric IL-2 on its surface. Infection of this cell line with influenza virus resulted in the incorporation of bioactive IL-2 on virus particles, even after viral inactivation. These results illustrate that membrane-bound cytokines can be stably packaged into virus particles and retain bioactivity upon viral inactivation.

In addition, the mouse specific IL2 cytokine has also been constructed in the same fashion and fused to the cytoplasmic and transmembrane domains of the viral neuraminidase protein. Further, as demonstrated in the Examples section, infra, two constructs comprising the gene coding for mature mouse GM-CSF linked to the gene fragment coding for the carboxy terminus of the hemagglutinin (HA) gene of influenza virus, i.e., a gene segment that codes for the last 71 or 43 amino acids of the HA protein and comprises the intracytoplasmic domain, the transmembrane domain and a variable stalk extracellular domain of HA, have been produced.

In another embodiment, the present invention also includes the production of viruses with immunomodulatory proteins linked to multiple envelope protein serotypes. For example, multiple HA and/or NA serotypes together with immunomodulatory proteins can be co-expressed and presented in inactivated viral vaccines. H1, H3 and N2, N1 antigens have been incorporated in the same viral filaments using dual infections. Incorporating multiple envelope protein serotypes together with cytokines may enhance heterotypic humoral and cellular immunity.

It is understood that the present invention is not limited to the use of influenza viruses. Viruses useful in the present invention include any enveloped virus, including, but not limited to, viruses belonging to the Orthomyxoviridae, Herpesviridae, Poxyiridae, Hepadnaviridae, Coronaviridae, Flaviviridae, Togaviridae, Retroviridae, Filoviridae, Paramyxoviridae, Rhabdovirisae, Arenaviridae, and Bunyaviridae virus families. Examples of viruses for use in the invention include, but are not limited to, influenza viruses, e.g., human and avian influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies. Therefore, the present invention provides wide application for the production and use of virus vaccines having augmented, e.g., enhanced and/or extended immunogenicity due to envelope-bound immunomodulatory proteins. The present invention can be combined with any existing technology whereby viral expression systems are used to make viral vaccines, including, but not limited to, the use of viral vectors to present tumor antigens to the immune system. The present invention also enhances the efficacy of current viral vectors that display tumor-associated antigens (TAA's) on their surface or in context with viral antigens.

### DEFINITIONS

As used herein, an “immune response” has the ordinary meaning in the art and, unless otherwise specified, refers to innate immunity or an adaptive immune response to a specific antigen. In one aspect, an immune response involves the action of lymphocytes, antigen presenting cells, phagocytic cells, and various soluble macromolecules in defending the body against infection, or other exposure to non-self molecules. The immune response can be detected and quantified (e.g., following immunization) by measuring cellular or humoral responses according to numerous assays known in the art (see, e.g., Coligan et al., 1991 (suppl. 1999), CURRENT PROTOCOLS IN IMMUNOLOGY, John Wiley & Sons). For example, to detect a cellular immune response, T cell effector effects against cells expressing the antigen are detected using standard assays, e.g., target-cell killing, lymphocyte proliferation, macrophage activation, B-cell activation or lymphokine production. Humoral responses are measured by detecting the appearance of, or increase in titer of, antigen-specific antibodies using routine methods such as ELISA. The progress of the antibody response can be determined by measuring class switching (i.e., the switch from an early IgM response to a later IgG response).

The terms “adjuvant” and “immunoadjuvant,” used interchangeably herein, refer to a compound or molecule that augments the host's immune response to an antigen when administered with that antigen. Adjuvant-mediated enhancement and/or extension of the duration of the antigen-specific immune response can be assessed by any method known in the art including, without limitation, an increase in a humoral or cellular immune response, e.g., a cytotoxic T cell or helper T cell immune response.

Adjuvants of the invention include immunomodulatory proteins, or portions thereof having activity as an adjuvant.

As used herein, the term “immunomodulatory” means that an agent, e.g., a protein or peptide, is capable of enhancing a humoral and/or cellular immune response, e.g., a cytotoxic T cell response or a T helper cell response, when administered to an animal having an immune system. An immunomodulatory protein includes any protein, or active portion thereof, having the ability to induce, enhance, or extend the immune response of a host. In a preferred embodiment, an immunomodulatory protein is a cytokine, chemokine or costimulatory molecule. In one embodiment, the immunomodulatory protein originates from a source foreign to the particular host cell or genome, e.g., viral genome.

The term “cytokine” as used herein, includes the general class of proteins secreted by cells of the immune system that serve to mediate and regulate immunity, inflammation, and hematopoiesis. Lymphokines, chemokines, monokines, interferons, colony-stimulating factors, and tumor necrosis factors are non-limiting examples of cytokines. The definition is meant to include, but is not limited to, those cytokines that, when used in accordance with the present invention, will result in alteration, e.g., inducing, enhancing or extending, of an individual's immune response. The cytokine can be, but is not limited to, IL-1α or IL-1β, IL-2, IL-3, IL-4, IL-5, IL-6, IL-7, IL-8, IL-9, IL-10, IL-11, IL-12, IL-15, IL-18, GM-CSF, M-CSF, G-CSF, LIF, LT, TGF-β, γ-IFN, IFNα or IFNβ, TNFα, BCGF, CD2, or ICAM. Descriptions of the aforementioned cytokines as well as other immunomodulatory agents may be found in “Cytokines and Cytokine Receptors”, A. S. Hamblin, 1993, (D. Male, ed., Oxford University Press, New York, N.Y.), or the “Guidebook to Cytokines and Their Receptors”, 1995, N. A. Nicola, ed. (Oxford University Press, New York, N.Y.) herein incorporated by reference.

As used herein, the term “chemokine” refers to a class of cytokines that play an important role in inflammatory responses, leukocyte trafficking, angiogenesis, and other biological processes related to the migration and activation of cells. As mediators of chemotaxis and inflammation, chemokines play roles in pathological conditions. Known chemokines are typically assigned to one of four subfamilies based on the arrangement of cysteine motifs and include: the alpha-chemokines, the beta-chemokines, the gamma chemokines and the delta-chemokines. For a recent review on chemokines, see Ward et al., 1998, Immunity 9:1-11 and Baggiolini et al., 1998, Nature 392:565-568, and the references cited therein. Chemokine activity may be mediated by chemokine receptors. For example, several seven-transmembrane-domain G protein-coupled receptors for C—C chemokines have been cloned: a C—C chemokine receptor-1 which recognizes MIP-1α, RANTES, MCP-2, MCP-3, and MIP-5 (Neote et al., 1993, Cell, 72:415-415); CCR2 which is a receptor for MCP1, 2, 3 and 4 or 5; CCR3 which is a receptor for RANTES, MCP-2, 3, 4, MIP-5 and eotaxin; CCR5 which is a receptor for MIP-1α, MIP-1β and RANTES; CCR4 which is a receptor for CMDC or TARC; CCR6 which is a receptor for LARC; and CCR7 which is a receptor for SLC and MIP-3 (reviewed in Sallusto et al., 1998, Immunol. Today 19:568 and Ward et al., 1998, Immunity 9:1-11). IL-8 is a chemokine that has been used to augment immune responses (Sin J, Kim J J, Pachuk C, Satishchandran C, Weiner D B. J. Virol. 2000 December; 74(23):11173-80).

As used herein, the term “costimulatory molecule” includes molecules which interact with a T cell which has received a primary activation signal to regulate T cell proliferative response and induction of effector functions. Costimulatory molecules are described in, for example, U.S. Pat. No. 6,294,660, incorporated herein by reference. Examples of costimulatory molecules include, but are not limited to CD80, CD86, ICAM-1, LFA-3, C3d, CD40L and Flt3L.

The term “vaccine” refers to a composition, e.g., a live vaccine or an inactivated virus vaccine, including a whole inactivated virus or a inactivated subunit, that can be used to elicit protective immunity in a recipient. It should be noted that to be effective, a vaccine of the invention can elicit immunity in a portion of the immunized population, as some individuals may fail to mount a robust or protective immune response, or, in some cases, any immune response. This inability may stem from the individual's genetic background or because of an immunodeficiency condition (either acquired or congenital) or immunosuppression (e.g., due to treatment with chemotherapy or use of immunosuppressive drugs). Vaccine efficacy can be established in animal models.

The phrase “inactivated virus” as used herein, refers to a virus that is no longer able to replicate. However, upon administration to a subject, the virus is still able to stimulate an immune response. Inactivated virus vaccines can be produced from the whole virus or the virus can be disrupted and only subunits of the virus used in the vaccine. Vaccines produced from the whole virus are referred to as “inactivated whole virus vaccines”, while vaccines using subunits of disrupted viruses are referred to as “inactivated subunit vaccines.” Inactivation of viruses can be carried out by any method known in the art including, without limitation, the methods described in the Examples section. In a preferred embodiment, a method for inactivation of a virus is one that does not reduce the functional activity of an immunomodulatory protein bound to, i.e., expressed by, the virus. Inactivated whole virus vaccines as well as inactivated subunit vaccines can be used in the methods of the invention, e.g., inactivated subunit vaccines which retain active immunostimulatory protein active after virus inactivation.

As used herein, the terms “vaccine” and “virus vaccine” also include “virus-like particle vaccines”. Virus-like particle vaccines that include membrane-bound immunomodulatory molecules are also included in the present invention. As used herein, the term “virus-like particle” is an assembly of capsid proteins into a shell-like structure without nucleic acid. Therefore, virus-like particles are non-infectious. These empty shells can display conformational epitopes that are not present on individual purified capsid proteins.

The term “DNA vaccine” is an informal term of art, and is used herein to refer to a vaccine delivered by means of a recombinant vector. An alternative, and more descriptive term used herein is “vector vaccine” (since some potential vectors, such as retroviruses and lentiviruses are RNA viruses, and since in some instances non-viral RNA instead of DNA is delivered to cells through the vector).

As used herein, the term “enveloped virus” includes any virus that has an outer envelope (also referred to herein as a “viral membrane”). Enveloped viruses obtain their envelope during maturation, in a process referred to as “budding” through a host cell membrane. Some viruses bud through specialized parts of the plasma membrane of the host cell or from internal membranes, such as the nuclear, endoplasmic reticulum or golgi compartmental membranes. The viral envelope is made up of carbohydrates, lipids, and proteins. The lipids and carbohydrates of the viral envelope are derived directly from the host cell, while the proteins in the envelope are virus-coded in most viruses (not all enveloped viruses exclude host cell proteins from incorporation).

During envelope assembly, virus-specified envelope proteins go directly to the appropriate cell membrane, displacing host proteins. The viral envelope has the lipid and carbohydrate constitution of the membrane where its assembly takes place. A given virus will differ in its lipids and carbohydrates when grown in different cells, with consequent differences in physical, biological, and antigenic properties. Viruses, including enveloped viruses are described in detail in “Fields-Virology,” Fields Virology, Fourth Edition, volumes 1 and 2 ed. Knipe and Howley, incorporated herein by reference.

The term “viral envelope protein” or “envelope protein,” includes glycoproteins and proteins contained within and that span the viral envelope (transmembrane proteins). Matrix proteins are non-glycosylated and are found as a layer on the inside of the envelope of virions of several viral families and provide added rigidity to the virion. Some enveloped viruses, including arenaviruses, bunyaviruses, and coronaviruses, have no matrix protein. In a preferred embodiment, the present invention is carried out using viral envelope glycoproteins, or fragments thereof. In one embodiment, the immunomodulatory proteins used in the invention are linked to amino terminus fragment of an envelope glycoprotein, e.g., the amino terminus of the glycoprotein, which comprises the transmembrane domain, the cytoplasmic domain and a linker or extracellular stalk domain (e.g., type I transmembrane protein). In another embodiment, the immunomodulatory protein is linked to the carboxy terminus fragment comprising the transmembrane domain, the cytoplasmic domain, and/or a fragment of the stalk domain of the glycoprotein (e.g., type II transmembrane protein). Examples of viral envelope glycoproteins for use in the present invention include, but are not limited to neuraminidase (NA), hemagglutinin (HA) hemagglutinin-neuraminidase (HN) or hemagglutinin-esterase-fusion (HEF) glycoproteins from viruses belonging to the orthomyxoviridae. Other specific embodiments would include viral envelope glycoproteins, including but not limited to E2E1, E, gp62 (Togaviridae); EFPgp64 (Baculoviridae); HN, F, H or G (Parainyxoviridae), G (Rhabdoviridae); gp41, gp37, p15E, gp36, gp22, gp30, gp48 (Retroviridae); E, HE, S, M (Coronoviridae); G1/G2 (Bunyaviridae); gG, gE, gI, gD, gJ, gK, gC, gB, gH, gM, gL, gp85/BXLF2, gp25/BKRF2, gp110 BALF4, gp84/113/BBRF3, gp15/BLRF1 and gp 350/220 (Herpesviridae) A33R, A34R, A36R, A56R, B5R, H3L, 15L and A27L (Poxyiridae); GP (Filoviridae) G1, gPr90, gp51, gp52, gp55, gp70, gp120 and gp41, and E1 and E2, and any other known glycoproteins including those described in Fields Virology, Fourth Edition, volumes 1 and 2 ed. Knipe and Howley. The present invention also includes various serotypes of the glycoproteins described herein.

Enveloped viruses useful in the present invention include those viruses belonging to, for example, any one of the following families of viruses: Orthomyxoviridae, Herpesviridae, Poxyiridae, Hepadnaviridae, Coronaviridae, Flaviviridae, Togaviridae, Retroviridae, Filoviridae, Paramyxoviridae, Rhabdovirisae, Arenaviridae, and Bunyaviridae, and Baculoviridae. For example, viruses used in the invention include, but are not limited to, influenza viruses including human and avian influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

The term “retrovirus” as used herein, is a class of enveloped viruses, belonging to the Retroviridae family, that have their genetic material in the form of RNA and use the reverse transcriptase enzyme to translate their RNA into DNA in the host cell. Many cancers in vertebrates are caused by retroviruses. Examples of retroviruses include HIV, HTLV-1, Mouse mammary tumor virus, Avian leukosis virus, Murine leukemia virus, Bovine leukemia virus, and Walley dermal sarcoma virus.

The term “linked” refers to the direct or indirect fusion of one protein to a second protein. For example, the term “linked” when used in the phrase “immunomodulatory protein linked to a viral envelope protein” refers to the direct or indirect fusion of an immunomodulatory protein, or a portion thereof, to a viral envelope protein, or a portion thereof. Linkage of an immunomodulatory protein, or a portion thereof, to a viral envelope protein, or a portion thereof may be carried out, for example, by producing a construct comprising a nucleotide sequence encoding an immunomodulatory protein, or a portion thereof, and a viral envelope protein, or a portion thereof. The construct may be contained within a vector, e.g., an expression vector. Once expressed, the proteins are fused to each other resulting in a single expression product. In another embodiment, an immunomodulatory protein, or a portion thereof, is indirectly linked to a viral envelope protein, or a portion thereof, e.g., using a linker.

The term “envelope-bound,” used interchangeably herein with “membrane-bound,” refers to a molecule that is bound, attached, or tethered, either directly or indirectly, to the envelope of a virus or a membrane of a cell, e.g., an animal cell. In one embodiment, the molecule, e.g., immunomodulatory protein, is bound to an envelope or membrane via a glycoprotein of the envelope or membrane. For example, the molecule, e.g., immunomodulatory protein, is linked or fused to a glycoprotein which is contained within the envelope or membrane.

The term “antigen” refers to any agent (e.g., protein, peptide, glycoprotein, glycolipid, nucleic acid, or combination thereof) that, when introduced into a host, animal or human, having an immune system, is capable of eliciting an immune response. As defined herein, the antigen-induced immune response can be humoral or cell-mediated, or both. An agent is termed “antigenic” when it is capable of specifically interacting with an antigen recognition molecule of the immune system, such as an immunoglobulin (antibody) or T-cell antigen receptor (TCR).

As used herein, the term “native antibodies” or “immunoglobulins” refers to usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain (VL) at one end and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 1985; 186: 651-663; Novotny and Haber, Proc. Natl. Acad. Sci. USA 1985; 82: 4592-4596).

The term “antibody” or “Ab” is used in the broadest sense and specifically covers not only native antibodies but also single monoclonal antibodies (including agonist and antagonist antibodies), antibody compositions with polyepitopic specificity, as well as antibody fragments (e.g., Fab, F(ab′)₂ scFᵥ and Fᵥ), so long as they exhibit the desired biological activity.

An “immunogenic amount” of a compound, agent or composition is an amount sufficient to induce an immune response in a host animal when administered to the host.

The terms “priming” or “primary” and “boost” or “boosting” are used herein to refer to the initial and subsequent immunizations, respectively, i.e., in accordance with the definitions as commonly used.

The term “subject” as used herein refers to an animal having an immune system, preferably an animal (e.g., a rodent such as a mouse, or an agriculturally important livestock such as a cow, pig, poultry, e.g., chicken, duck, goose, turkey, or other animal, e.g., a horse, sheep, dog, cat, monkey, or rabbit). In particular, the term refers to humans.

The term “epitope” or “antigenic determinant” refers to any portion of an antigen recognized either by B cells, or T-cells, or both. Preferably, interaction of such epitope with an antigen recognition site of an immunoglobulin (antibody) or T-cell antigen receptor (TCR) leads to the induction of antigen-specific immune response. T-cells recognize proteins only when they have been cleaved into smaller peptides and are presented as a complex with MHC molecules located on another cell's surface.

The term “treat” is used herein to mean to relieve or alleviate at least one symptom of a disease in a subject. Within the meaning of the present invention, the term “treat” may also mean to prolong the prepatency, i.e., the period between infection and clinical manifestation of a disease. The term “protect” is used herein to mean prevent or treat, or both, as appropriate, development or continuance of a disease in a subject. In one embodiment, the disease is an infectious disease, e.g., a viral infection caused by, for example, an influenza virus, e.g., a human or avian influenza virus, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

The term “protective immunity” refers to an immune response in a host animal (either active/acquired or passive/innate, or both) which leads to inactivation and/or reduction in the load of said antigen and to generation of long-lasting immunity (that is acquired, e.g., through production of antibodies), which prevents or delays the development of a disease upon repeated exposure to the same or a related antigen. A “protective immune response” comprises a humoral (antibody) immunity or cellular immunity, or both, effective to, e.g., eliminate or reduce the load of a pathogen or infected cell (or produce any other measurable alleviation of the infection) in an immunized (vaccinated) subject.

As used herein, the term “augment an immune response” or “augment an immunogenicity” refers to enhancing or extending the duration of an immune response, or both. When referred to a property of an agent (e.g., an adjuvant), the term “[able to] augment the immunogenicity of an antigen” refers to the ability to enhance the immunogenicity of an antigen or the ability to extend the duration of the immune response to an antigen, or both.

The phrase “enhance immune response” within the meaning of the present invention refers to the property or process of increasing the scale and/or efficiency of immunoreactivity to a given antigen. When used in reference to the immunomodulatory proteins used in the invention, said immunoreactivity is either humoral or cellular immunity, e.g., CD4+ and/or CD8+ T cell-mediated immunity. An immune response is believed to be enhanced, if any measurable parameter of antigen-specific immunoreactivity (e.g., T-cell production or antibody production) is increased at least two-fold, five-fold, preferably ten-fold, most preferably twenty-fold or thirty-fold.

The term “therapeutically effective” applied to dose or amount refers to that quantity of a compound or pharmaceutical composition or vaccine that is sufficient to result in a desired activity upon administration to an animal in need thereof. As used herein with respect to the virus vaccines of the invention, the term “therapeutically effective amount/dose” is used interchangeably with the term “immunogenically effective amount/dose” and refers to the amount/dose that is sufficient to produce an effective immune response upon administration to an animal. According to the present invention, a preferred immunogenically effective amount of the virus vaccine of the invention is in the range of 0.001 to 1.0 mg per kg of body weight.

The phrase “pharmaceutically acceptable”, as used in connection with compositions of the invention, refers to molecular entities and other ingredients of such compositions that are physiologically tolerable and do not typically produce unwanted reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

The term “carrier” applied to pharmaceutical or vaccine compositions of the invention refers to a diluent, excipient, or vehicle with which a compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution, saline solutions, and aqueous dextrose and glycerol solutions are preferably employed as carriers, particularly for injectable solutions. Suitable pharmaceutical carriers are described in “Remington's Pharmaceutical Sciences” by E. W. Martin, 18th Edition.

The term “about” or “approximately” usually means within 20%, more preferably within 10%, and most preferably still within 5% of a given value or range. Alternatively, especially in biological systems (e.g., when measuring an immune response), the term “about” means within about a log (i.e., an order of magnitude) preferably within a factor of two of a given value.

The terms “vector”, “cloning vector”, and “expression vector” mean the vehicle by which a DNA or RNA sequence (e.g., a foreign gene) can be introduced into a host-cell. These vehicles may also promote expression (e.g., transcription and/or translation) of the introduced sequence in a host cell. Vectors include plasmids, phages, viruses, etc.

In accordance with the present invention, conventional molecular biology, microbiology, and recombinant DNA techniques may be employed within the skill of the art. Such techniques are well-known and are explained fully in the literature. See, e.g., Sambrook, Fritsch and Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

A “nucleic acid molecule” (or alternatively “nucleic acid”) refers to the phosphate ester polymeric form of ribonucleosides (adenosine, guanosine, uridine, or cytidine: “RNA molecules”) or deoxyribonucleosides (deoxyadenosine, deoxyguanosine, deoxythymidine, or deoxycytidine: “DNA molecules”), or any phosphoester analogs thereof, such as phosphorothioates and thioesters, in either single stranded form, or a double-stranded helix. Oligonucleotides (having fewer than 100 nucleotide constituent units) or polynucleotides are included within the defined term as well as double stranded DNA-DNA, DNA-RNA, and RNA-RNA helices. This term, for instance, includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

As used herein, the term “protein” refers to an amino acid-based polymer, which can be encoded by a nucleic acid or prepared synthetically. Proteins include protein fragments, chimeric proteins, etc. Generally, a DNA sequence encoding a particular protein or enzyme is “transcribed” into a corresponding sequence of mRNA. The mRNA sequence is, in turn, “translated” into the sequence of amino acids which form a protein. An “amino acid sequence” is any chain of two or more amino acids. The term “peptide” is usually used for amino acid-based polymers having fewer than 100 amino acid constituent units, whereas the term “polypeptide” is reserved for polymers having at least 100 such units.

Use of the Viral Vaccines of the Invention

In one aspect, the present invention provides a method for inducing an immune response in an animal comprising administering to the animal an effective amount of a composition comprising an inactivated virus expressing an envelope-bound immunomodulatory protein, wherein the immune response induced by the animal is more robust, e.g., enhanced or extended, as compared to the immune response that could have been induced in the animal by the virus without the envelope-bound immunomodulatory protein.

The use of an envelope-bound immunomodulatory protein to mediate activation of the immune system has distinct advantages. Administered alone, immunomodulatory proteins, e.g., cytokines and chemokines are soluble proteins with short half lives and quickly diffuse from the injection site, thereby reducing their effectiveness as adjuvants and/or inducing toxicity in the subject. Anchoring of immunomodulatory proteins, e.g., cytokines, directly to virus envelopes allows for close proximity of the immunomodulatory protein and the antigen upon delivery to the subject and prevents diffusion from the site of injection, to thereby increase the effectiveness of the vaccine composition and reduce toxicity to the subject. Administration of the viruses described herein also results in broader vaccine efficacy, e.g., protection against the targeted viral strain as well as variants thereof. Furthermore, administration of the viruses expressing envelope-bound immunomodulatory proteins, as described herein, allows for lower doses of virus per vaccine. Among other benefits, this would reduce the time needed to produce adequate amounts of vaccine for use against viral variants, pandemics, e.g., avian influenza pandemics, and bioterrorist introduction of virus.

Reducing viral load is important in preventing the emergence of highly pathogenic strains of viruses, for example avian influenza. Vaccines that induce robust, e.g., enhanced or extended, humoral and/or cellular responses such as the vaccines of the present invention will also reduce overall disease severity and the load of virus circulating in the subject.

The immune response induced by the virus vaccines described herein may be a humoral immune response and/or a cellular immune response. The terms “humoral immunity” or “humoral immune response” are meant to refer to the form of acquired immunity in which antibody molecules are secreted in response antigenic stimulation. The terms “cell-mediated immunity” and “cell-mediated immune response” are meant to refer to the immunological defense provided by lymphocytes, such as that defense provided by T cell lymphocytes when they come into close proximity to their victim cells. A cell-mediated immune response also comprises lymphocyte proliferation. When “lymphocyte proliferation” is measured, the ability of lymphocytes to proliferate in response to specific antigen is measured. Lymphocyte proliferation is meant to refer to B cell, T-helper cell or CTL cell proliferation. In one embodiment, the immune response induced by the virus vaccine is a cytotoxic T cell immune response and/or a helper T cell immune response. Immune response can be determined using assays known in the art. For example, the presence of antigen primed-T helper cells can be detected using lymphocyte proliferation assays as described herein and in Hu et al. (2001) (Current Progress on Avian Immunology Research, ed. K. A. Schat. American Association Avian Pathologists: Kennett Square. 269-274). An assay useful for determining T cell cytotoxicity is also described herein and in Seo and Webster, J. Virol. 2001; 75(6): 2516-25.

In another aspect, the present invention provides methods for treating or preventing a viral infection in an animal comprising administering to the animal an inactive, enveloped virus expressing an envelope-bound immunomodulatory protein. Viral infections that may be treated or prevented by the methods of the invention include those infections caused by any enveloped virus. For example, in one embodiment, the virus belongs to the family of viruses selected from the group consisting of Orthomyxoviridae, Herpesviridae, Poxyiridae, Hepadnaviridae, Coronaviridae, Flaviviridae, Togaviridae, Retroviridae, Filoviridae, Paramyxoviridae, Rhabdovirisae, Arenaviridae, Baculoviridae and Bunyaviridae. In another embodiment, the virus is selected from the group consisting of influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies. Viral infections which may be treated by the methods of the invention are by no means limited to infections caused by the examples listed above.

According to the present invention, an inactivated virus expressing an envelope-bound immunomodulatory protein may be administered to a subject by any means that results in an immune response in the subject, including, for example, intramuscular (i.m.), intradermal (i.d.), intranasal, subcutaneous (s.c.), and oral. A preferred immunogenically effective amount of an inactivated virus expressing an envelope-bound immunomodulatory protein of the invention is in the range of 0.001 mg-1 mg per kg of body weight.

The method of the invention can be practiced in any animal. In a specific embodiment, the animal is human. In another specific embodiment, the animal is, e.g., a rodent such as a mouse, or an agriculturally important livestock such as a cow, pigs, poultry, or other animal, e.g., a horse, sheep, monkey, dog, cat or rabbit.

Immunomodulatory Proteins

Immunomodulatory proteins that are useful in the invention include any protein or peptide that is capable of augmenting, e.g., enhancing a humoral and/or cellular immune response, e.g., a cytotoxic T cell response or a T helper cell response, when administered to an animal having an immune system. In a preferred embodiment, an immunomodulatory protein is a cytokine, chemokine or costimulatory molecule. Examples of cytokines that may be used in the invention include, but are not limited to, IL-1α or IL-1β, IL-2, IL-3, IL-4, IL-5, IL-6, IL-7, IL-8, IL-9, IL-10, IL-11, IL-12, IL-15, IL-18 GM-CSF, M-CSF, G-CSF, LIF, LT, TGF-β, γ-IFN, IFNα or IFNβ, TNFα, BCGF, CD2, or ICAM. Examples of chemokines that may be used in the invention include, but are not limited to, IL-8, SDF-1α, MCP1, 2, 3 and 4 or 5, RANTES, MIP-5, MIP-3, eotaxin, MIP-1α, MIP-1β, CMDC, TARC, LARC, and SLC. Examples of costimulatory molecules that my be used in the invention are CD80, CD86, ICAM-1, LFA-3, C3d, CD40L and Flt3L.

Where use of the invention in humans is contemplated, immunomodulatory protein, e.g., the cytokine, chemokine or costimulatory molecule will preferably be substantially similar to the human form of the protein or have been derived from human sequences (i.e., of human origin). Similarly, when use in another animal is contemplated, the cytokine, chemokine or costimulatory molecule will preferably be substantially similar to the form of the protein of the corresponding animal or derived from that animal. Additionally, cytokines, chemokines or costimulatory molecules of other animals with substantial homology to the human forms of, for example, IL-2, and others, will be useful in the invention when demonstrated to exhibit similar activity on the immune system. Similarly, proteins that are substantially analogous to any particular cytokine, chemokine or costimulatory molecule, but have relatively minor changes of protein sequence, will also find use in the present invention. It is well known that some small alterations in protein sequence may be possible without disturbing the functional abilities of the protein molecule, and thus proteins can be made that function as cytokines, chemokines or costimulatory molecules in the present invention but differ slightly from currently known sequences. Thus, proteins that are substantially similar to any particular cytokine, chemokine or costimulatory molecule will also find use in the present invention. As used herein, two DNA sequences are “substantially homologous” or “substantially similar” when at least about 80%, and most preferably at least about 90 or 95%, 96%, 97%, 98%, or 99% of the nucleotides match over the defined length of the DNA sequences, as determined by sequence comparison algorithms, such as BLAST, FASTA, DNA Strider, etc. An example of such a sequence is an allelic or species variant of the a gene encoding an immunomodulatory protein used in the present invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 70%, 75%, 80%, 85%, 86%, 87%, 88%, 89%, 90%, 90%, 91%, 92%, 93%, 94%, 95%, 96%, 97%, 98%, or 99% of the amino acids are identical, or greater than about 90%, 91%, 92%, 93%, 94%, 95%, 96%, 97%, 98%, or 99% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

Production of Viral Vaccines of the Invention

The present invention provides methods for producing an enveloped virus expressing an envelope-bound, immunomodulatory protein as well as cell lines which stably express the immunomodulatory protein on the surface of the cell. To produce the viruses of the invention which comprise immunomodulatory proteins bound to enveloped viruses, immunomodulatory molecules, e.g., mature cytokines or chemokines, are linked to viral envelope proteins, e.g., glycoproteins and displayed on the surface of cells which are then infected with virus. For example, constructs comprising a nucleic acid molecule encoding an immunomodulatory protein, or portion thereof linked to an envelope protein, or portion thereof, may be introduced into a vector and expressed in a cell. Any cell that allows productive virus replication, such as, for example, but not limited to, a Madin-Darby Canine Kidney (MDCK) cell, VERO cells, an African green monkey kidney cell line, or BHK (baby hamster kidney cells) or derivatives thereof, may be used in the invention. Transfection of cells with an expression vector comprising an immunomodulatory protein, or portion thereof, and an envelope protein, or portion thereof, results in the display of the immunomodulatory protein on the surface of the cell or other internal cell membrane. Thus, the present invention includes methods for producing cell lines that stably and constitutively express specific immunomodulatory proteins, e.g., cytokine, chemokine or costimulatory molecule, bound to viral envelope proteins. Once infected with a virus, i.e., an enveloped virus, these cell lines are used to produce viruses expressing the immunomodulatory protein bound (tethered) to the viral envelope protein.

Viral envelope proteins that may be used in the methods of the invention include envelope proteins derived from any enveloped virus. In a preferred embodiment, the viral envelope protein used in the methods of the invention is a glycoprotein. In one embodiment, a portion of the viral enveloped protein is fused to the immunomodulatory protein. For example, in one embodiment, the portion of the envelope protein fused to the immunomodulatory protein includes the cytoplasmic domain and the transmembrane domain of the envelope protein. In yet another embodiment, the portion of the envelope protein fused to the immunomodulatory protein includes amino acids of the stalk domain of the envelope protein as well as the cytoplasmic domain and/or the transmembrane domain of the protein. In yet another embodiment linker amino acids with a rigid structure are incorporated between the immunomodulatory protein and/or amino acids of the stalk of the envelope protein as well as the cytoplasmic domain and/or the transmembrane domain of the protein.

In one embodiment, a vaccine virus of the invention is produced by inactivating the virus, e.g., the whole virus or subunits thereof. Any method known in the art or described herein may be used to inactivate the virus. For example, methods for virus inactivation include use of beta-propiolactone, as described in Budowsky, E. I., A. Smirnov Yu, and S. F. Shenderovich, Vaccine 1993; 11(3):343-8; heat, as described in Cho, Y., et al., J. Virol. 2003; 77(8):4679-84; formalin, as described in Lu, X., et al., J. Virol. 2001; 75(10):4896-901; and UV radiation, as described in Moran, T. M., et al., J. Infect. Dis. 1999; 180(3):579-85. Additional methods for virus inactivation are described in, for example, U.S. Pat. No. 6,136,321.

In a preferred embodiment, the virus is inactivated while the immunomodulatory protein retains its activity, e.g., the ability to augment, e.g., enhance an immune response. Methods for determining the activity of an immunomodulatory protein, e.g., a cytokine, chemokine or costimulatory molecule, are known in the art and described herein.

### Formulations and Administration

In conjunction with the methods of the present invention, also provided are pharmaceutical and immunogenic compositions comprising an immunogenically effective amount of an inactivated virus vaccine comprising a virus expressing an envelope-bound immunomodulatory protein, which compositions are suitable for administration to induce an immune response for the treatment of and prevention of infectious diseases. Compositions of the present invention can be formulated in any conventional manner using one or more pharmaceutically acceptable carriers.

The vaccine compositions of the invention can be combined with other adjuvants and/or carriers. These other adjuvants include, but are not limited to, oil-emulsion and emulsifier-based adjuvants such as complete Freund's adjuvant, incomplete Freund's adjuvant, MF59, or SAF; mineral gels such as aluminum hydroxide (alum), aluminum phosphate or calcium phosphate; microbially-derived adjuvants such as cholera toxin (CT), pertussis toxin, Escherichia coli heat-labile toxin (LT), mutant toxins (e.g., LTK63 or LTR72), Bacille Calmette-Guerin (BCG), Corynebacterium parvum, DNA CpG motifs, muramyl dipeptide, or monophosphoryl lipid A; particulate adjuvants such as immunomodulatory complexes (ISCOMs), liposomes, biodegradable microspheres, or saponins (e.g., QS-21); cytokines such as IFN-γ, IL-2, IL-12 or GM-CSF; synthetic adjuvants such as nonionic block copolymers, muramyl peptide analogues (e.g., N-acetyl-muramyl-L-threonyl-D-isoglutamine [thr-MDP], N-acetyl-nor-muramyl-L-alanyl-D-isoglutamine, N-acetylmuramyl-L-alanyl-D-isoglutaminyl-L-alanine-2-[1′-2′-dipalmitoyl-sn-glycero-3-hydroxyphosphoryloxy]-ethylamine), polyphosphazenes, or synthetic polynucleotides, and surface active substances such as lysolecithin, pluronic polyols, polyanions, peptides, hydrocarbon emulsions, or keyhole limpet hemocyanins (KLH). Preferably, these additional adjuvants are also pharmaceutically acceptable for use in humans. The vaccine compositions of the vaccine can be combined with virus vaccine vectors expressing other antigenic epitopes, e.g., tumor associated antigens, TAAs, MHC class I or class II specific antigenic epitopes to augment their efficacy and enhance their immunogenicity.

Preferably, the vaccine formulations of the invention are delivered by subcutaneous (s.c.), intramuscular (i.m.), intradermal (i.d.), intranasal, or oral administration. Formulations for injection can be presented in unit dosage form, e.g., in ampoules or in multi-dose containers, with an added preservative. The compositions can take such forms as excipients, suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water, before use.

In addition to the formulations described previously, the compositions can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

As disclosed herein, the vaccine viruses can be mixed with pharmaceutically acceptable carriers. Suitable carriers are, for example, water, saline, buffered saline, dextrose, glycerol, ethanol, sterile isotonic aqueous buffer or the like and combinations thereof. In addition, if desired, the preparations may also include minor amounts of auxiliary substances such as wetting or emulsifying agents, pH buffering agents, and/or immune stimulators (e.g., adjuvants in addition to the immunomodulatory molecules expressed by the virus) that enhance the effectiveness of the pharmaceutical composition or vaccine. These additional immunomodulatory molecules can be delivered systemically or locally as proteins or by expression of a vector that codes for expression of the molecule or by any method known in the art.

The invention also provides a pharmaceutical pack or kit comprising one or more containers filled with one or more of the ingredients of the immunogenic formulations of the invention. In a related embodiment, the present invention provides a kit for the preparation of a immunogenic composition comprising a virus vaccine, and optionally instructions for administration of the viral vaccine. The kit may also optionally include one or more physiologically acceptable carriers and/or auxiliary substances. Associated with the kit can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device which may contain one or more unit dosage forms containing the active ingredient (i.e., a virus vaccine of the invention). The pack may, for example, comprise metal or plastic foil, such as a blister pack. The pack or dispenser device may be accompanied by instructions for administration. Compositions of the invention formulated in a compatible pharmaceutical carrier may also be prepared, placed in an appropriate container, and labeled for treatment of an indicated condition.

### Effective Dose and Safety Evaluations

According to the methods of the present invention, the pharmaceutical and immunogenic compositions described herein are administered to a patient at immunogenically effective doses, preferably, with minimal toxicity.

Following methodologies which are well-established in the art (see, e.g., reports on evaluation of several vaccine formulations containing novel adjuvants in a collaborative effort between the Center for Biological Evaluation and Food and Drug Administration and the National Institute of Allergy and Infectious Diseases [Goldenthal et al., National Cooperative Vaccine Development Working Group. AIDS Res. Hum. Retroviruses 1993, 9:545-9]), effective doses and toxicity of the compounds and compositions of the instant invention are first determined in preclinical studies using small animal models (e.g., chickens and mice) in which the virus vaccine has been found to be immunogenic and that can be reproducibly immunized by the same route proposed for the human clinical trials. Specifically, for any pharmaceutical composition or vaccine used in the methods of the invention, the therapeutically effective dose can be estimated initially from animal models to achieve a circulating plasma concentration range that includes the IC50 (i.e., the concentration of the test compound which achieves a half-maximal inhibition of symptoms). Dose-response curves derived from animal systems are then used to determine testing doses for the initial clinical studies in humans. In safety determinations for each composition, the dose and frequency of immunization should meet or exceed those anticipated for use in the clinical trial.

As disclosed herein, the dose of vaccine virus, and other components in the compositions of the present invention is determined to ensure that the dose administered continuously or intermittently will not exceed a certain amount in consideration of the results in test animals and the individual conditions of a patient. A specific dose naturally varies depending on the dosage procedure, the conditions of a patient or a subject animal such as age, body weight, sex, sensitivity, feed, dosage period, drugs used in combination, and seriousness of the disease. The appropriate dose and dosage times under certain conditions can be determined by the test based on the above-described indices and should be decided according to the judgment of the practitioner and each patient's circumstances according to standard clinical techniques. In this connection, the dose of a virus vaccine is generally in the range of between 0.0001 mg and 0.2 mg per kg of the body weight, preferably 0.02 to 0.2 mg per kg of the body weight of chickens and 0.00005 to 0.001 mg per kg of body weight for humans.

Toxicity and therapeutic efficacy of the virus vaccines of the invention can be determined by standard pharmaceutical procedures in experimental animals, e.g., by determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred. While therapeutics that exhibit toxic side effects can be used (e.g., life-threatening infections), care should be taken to design a delivery system that targets such immunogenic compositions to the specific site in order to minimize potential damage to other tissues and organs and, thereby, reduce side effects. In this respect, the advantage of the present invention is that, to exert the most potent effect, the vaccine is administered locally. As disclosed herein, the adjuvant of the invention, e.g., the viral envelope-bound cytokines or chemokines or costimmulatory molecules, are not only highly immunostimulating at relatively low doses but also possess low toxicity and does not produce significant side effects.

As specified above, the data obtained from the animal studies can be used in formulating a range of dosage for use in humans. The therapeutically effective dosage of the virus vaccines of the present invention for use in humans lies preferably within a range of circulating concentrations that include the ED50 with little or no toxicity. The dosage can vary within this range depending upon the dosage form employed and the route of administration utilized. Ideally, a single dose should be used.

### EXAMPLES

The following Examples illustrate the invention without limiting its scope.

### Example 1

### Preparation and Validation of Influenza Vaccines (IVACs) Bearing Immunomodulators

This Example illustrates the constitutive expression of biologically active chicken IL-2 fused to the amino terminus of influenza envelope protein neuraminidase (NA˜chIL2) in MDCK cells as well as the incorporation of NA˜chIL2 into filamentous viral particles budding from MDCK/NA˜chIL2 cells. Furthermore, this example demonstrates that virus particles bearing NA˜chIL2 retain IL-2 bioactivity following inactivation with UV radiation and heat, inactivation protocols described herein.

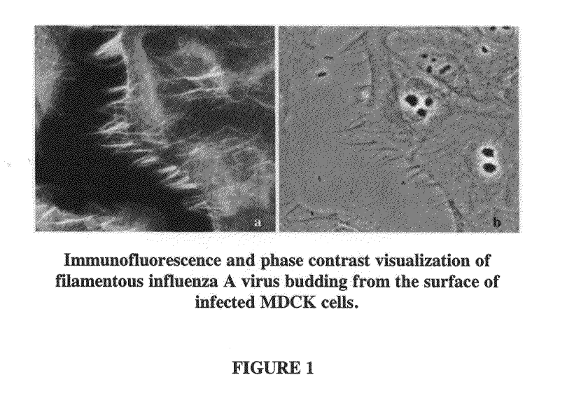

Influenza A/Udorn/72 is highly filamentous. In contrast to most laboratory-adapted strains of influenza virus which are found to produce virions of roughly spherical morphology and 100-150 nm diameter, the A/Udorn/72 strain of virus was found to produce a large number of long filamentous particles (FIG. 1). The filamentous influenza A/Udorn/72 virus was used for incorporation of avian immunomodulatory cytokines and chemokines directly into virus particles as described herein. The use of filamentous particles allowed for visual confirmation of avian cytokine incorporation using standard immuno-fluorescence staining techniques. Furthermore, the filamentous particle represents a large platform upon which multiple HA and NA serotypes together with immunostimulatory molecules can be co-expressed and presented in inactivated viral vaccines. Dual infections have been successfully employed in incorporating H1, H3 and N2, N1 antigens in the same viral filaments. Incorporating multiple HA serotypes together with avian or mouse or human cytokines may enhance heterotypic humoral and cellular immunity.

Constitutive Expression of NA˜chIL2 in MDCK cells. An expression plasmid was generated based on the commercially available pcDNA3.1 in which the coding region of chicken IL2 is fused to the N-terminus encoding region of the A/WSN neuraminidase gene. Thus, the bioactive COOH end of the chIL2 molecule was exposed extracellularly.

Using PCR, a NA˜chIL2 construct was made and inserted into the BamHI/EcoRI site present in the multiple cloning site of pcDNA3.1. This construct codes for a protein containing the N-terminal 6 amino acid cytoplasmic tail domain, the 29 amino acid transmembrane domain and the first 17 amino acids of the stalk region of the N1 protein fused to the mature chicken IL2 protein (minus the signal peptide). The N1 gene and protein are listed in Genbank Accession No. J02177 and are set forth herein as SEQ ID NO:1 and SEQ ID NO:2, respectively.

The chicken IL-2 gene and protein are listed as Genbank Accession No. AF000631 and in U.S. Pat. No. 6,190,901, and are set forth herein as SEQ ID NO:3 and SEQ ID NO:4, respectively.

The NA forward primer (5′ GAC TGG ATC CCT GCC ATG AAT CCA AAC-3′) (SEQ ID NO:5) codes for the BamHI enzyme (GGATCC) (SEQ ID NO:6) the chIL-2 Kozak sequence (CTGCC) (SEQ ID NO:7) and the first 12 nucleotides of the NA sequence.

The NA reverse primer (5′ A CT GCC TTG GTT GCA TAT 3′) (SEQ ID NO:8) encodes the STYI site (CCTTGG) (SEQ ID NO:9) present within the NA gene and 8 nucleotides upstream of that site.

The chIL-2 forward primer (5′ GCA TCC AAG GCG CAT CTC TAT CA 3′) (SEQ ID NO:10) encodes a STYI site (CCAAGG) (SEQ ID NO:11), a C to keep the fusion construct inframe and the first 12 nucleotides encoding mature chIL-2.

The chIL-2 reverse primer (5′ GCT AGA ATT CTT ATT TTT GCA 3′) (SEQ ID NO:12) encodes an ECORI site (GAATTC) (SEQ ID NO:13), stop codon (TTA) and the last 8 nucleotides of chIL-2. Standard PCR technology was used with the above primers and templates (full length genes for NA and chIL-2), respectively.

The two PCR products were cut with the respective restriction enzymes and ligated to each other and to the BamHI and EcoRI sites of pcDNA 3.1. The new plasmid construct, pcDNA3. INA-chIL-2, was transfected into MDCK cells (ATCC™) using Lipofectamine 2000 (Invitrogen™) as described by the manufacturers. The transfected cells were then selected for growth in G418 (1.5 mg/ml). Surviving cells were cloned by limiting dilution and screened for cell surface expression of NA˜chIL2 by standard immunofluorescence staining protocols using monoclonal antibodies specific to chicken IL-2.

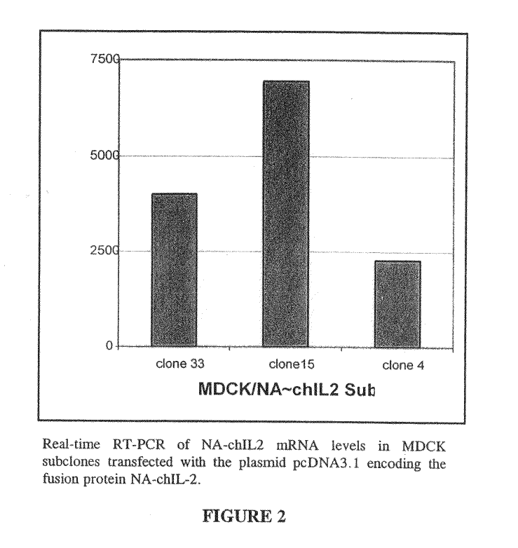

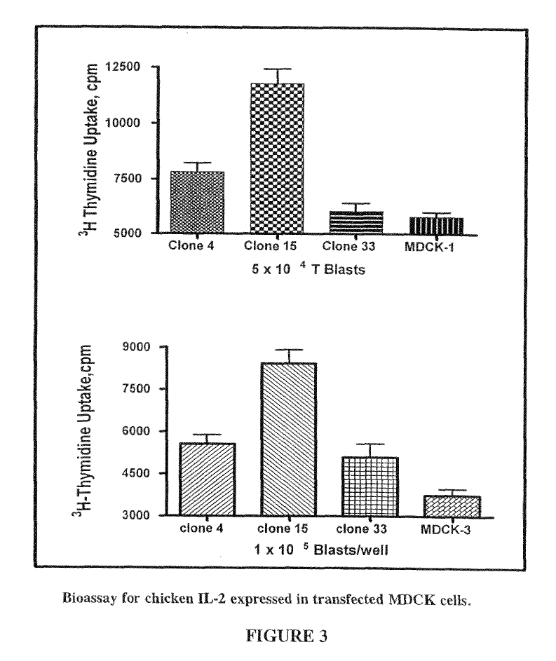

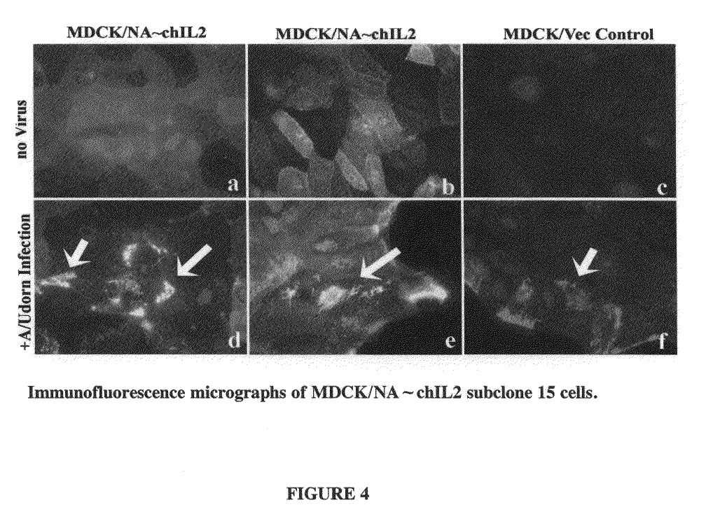

As depicted in FIG. 2, MDCK subclones were isolated which readily express chicken IL2 mRNA, as determined by real-time RT-PCR. In addition, it was confirmed that NA˜chIL2 was expressed at the cell surface (FIG. 4). In order to confirm that the NA˜chIL2 expressed at the cell surface of MDCK cells was biologically active, an in vitro bioassay (Sundick, R. S, and C. Gill-Dixon, J Immunol, 1997. 159(2): 720-5; Kolodsick, J. E., et al., Cytokine, 2001. 13(6): 17-24) was employed. Briefly, MDCK vector control cells or MDCK/NA˜chIL2 expressing cells were seeded in 96-well plates, grown to confluency, treated with mitomycin C for 1 hour (50 μg/ml), washed and incubated with different numbers of Con A-stimulated chicken T cell blasts for 24 hours (FIG. 3). During the final 6 hours, media were supplemented with 1 μCi ³H-thymidine and the amount of incorporation was determined in a liquid scintillation counter following harvesting. Clone 15 cells were able to significantly (p<0.01) stimulate T blasts compared to control MDCK cells (FIG. 3). Thus, this example shows that NA˜chIL2 expressed at the surface of MDCK cells is biologically active.

Incorporation of NA˜chIL2 into filamentous viral particles budding from MDCK/NA˜chIL2 sc.15 cells. The extent to which influenza virus incorporated NA˜chIL2 from the surface of MDCK cells was determined. It was possible to visualize incorporation directly on budding virus particles using immunofluorescence and phase contrast visualization (see FIG. 1). As depicted in FIG. 4 (d and e), NA˜chIL2 was readily incorporated into budding viral filaments projecting from the surface of MDCK infected cells. NA˜chIL2 was detected by labeling of viral filaments with monoclonal antibody specific for chIL2. Viral filaments budding from normal MDCK cells (vector control cells, FIG. 4f) did not stain positive with chIL2 antibody, confirming the specificity of the antibody for chIL2. This is the first report of incorporation of an avian-specific cytokine directly in an influenza virus particle, and illustrates the vaccine potential of an influenza virus vaccine bearing avian immunomodulators.

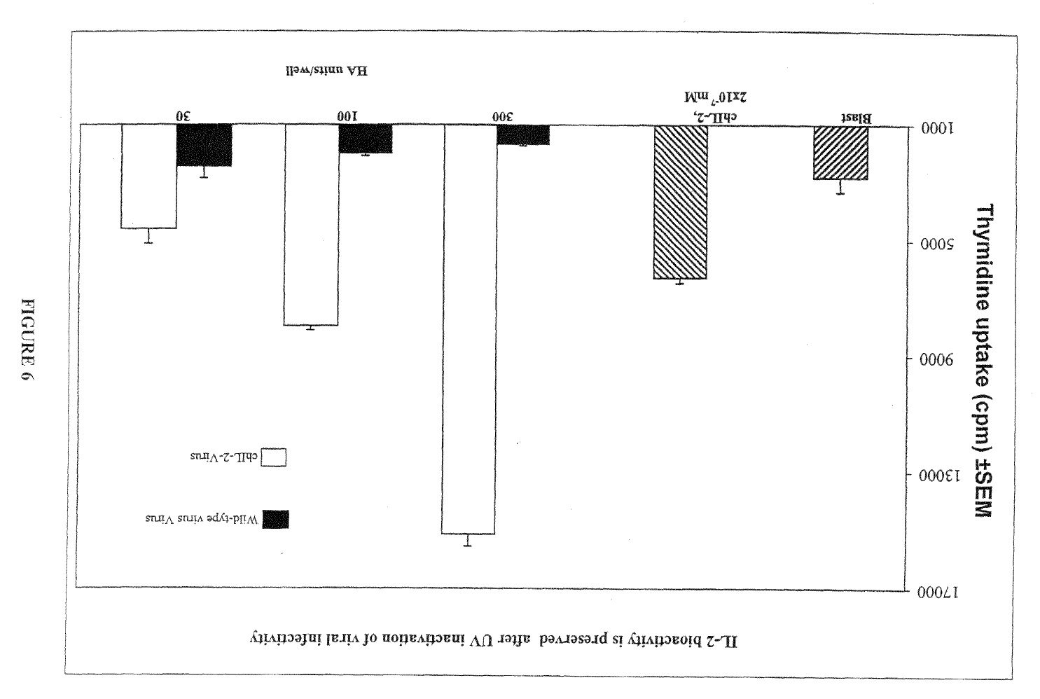

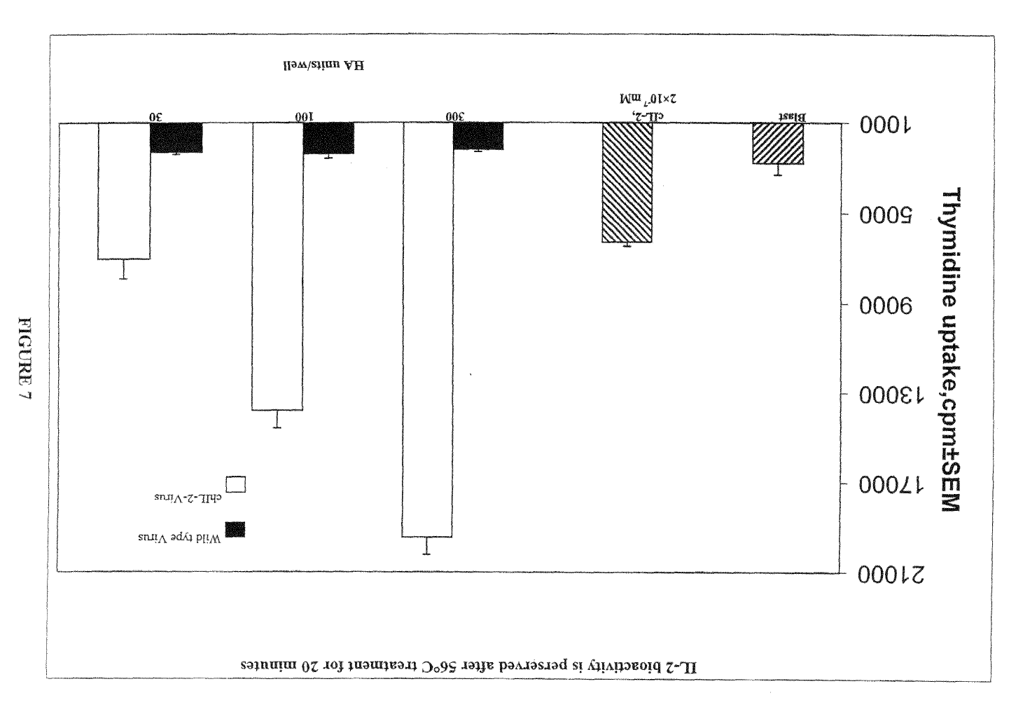

Membrane-bound cytokine bioactivity is preserved following inactivation of the virus by UV radiation and heat. Virus particles derived from A/Udorn infection of MDCK˜NAchIL2 (subline15 MDCK cells stably transfected with pcDNA3.1 encoding NA˜chIL2) or MDCK wildtype cells were harvested from culture supernatants and partially purified through a 14% Optiprep cushion. Following inactivation with UV radiation or heat (56° C. for 20 minutes), serial dilutions of inactivated virus-bearing chicken IL2 or conventional virus were used to stimulate proliferation of chicken T blasts as described (see FIG. 3). Using this bioassay, bioactivity was demonstrated using 30 HAU of chIL2-bearing influenza virus vaccine. FIG. 6 depicts bioactivity after inactivation with UV and FIG. 7 depicts bioactivity after inactivation with heat. Thus, membrane-bound cytokines expressed on virus particles retain bioactivity after viral inactivation. Using the bioassays described below, the vaccines will be standardized based on both HA antigenic content and immunomodulatory units per μg of HA.

In summary, these results illustrate that membrane-bound cytokines can be stably packaged into virus particles and retain bioactivity upon viral inactivation.

### Example 2

### Construction of Stable Cell Lines Constitutively Expressing Chicken Specific Cytokines Fused to Viral HA or NA

This Example illustrates the construction of stable cell lines constitutively expressing chicken specific cytokines fused to the cytoplasmic tail and transmembrane domains of viral HA or NA. Stable cell lines are assessed for stability of expression, retention of immunomodulatory activity and as a platform for incorporation into influenza virus particles.

Choice of Culture Platform for Vaccine Production. The use of animal cell culture is a viable substrate for propagation of influenza virus vaccines. An important factor for choosing between eggs and cell culture for vaccine propagation is the retention of vaccine antigenicity and potency. Subtle differences in antigenicity and in CTL responses have been reported for vaccine viruses propagated in eggs versus MDCK cells (Robertson, J. S., et al., J Gen Virol, 1991. 72 (Pt 11): 2671-7; Rocha, E. P., et al., J Gen Virol, 1993. 74 (Pt 11): 2513-8; Robertson, J. S., et al., Virology, 1990. 179(1): 35-40; Robertson, J. S., et al., Virology, 1987. 160(1): 31-7; Wood, J. M., et al., Virology, 1989. 171(1): 214-21; Wang, M. L., J. M. Katz, and R. G. Webster, Virology, 1989. 171(1): 275-9; Katz, J. M. and R. G. Webster, Virology, 1988. 165(2):446-56; Katz, J. M., M. Wang, and R. G. Webster, J Virol, 1990. 64(4): 1808-11; Katz, J. M. and J. S. Robertson, WHO-NIH meeting on host cell selection of influenza virus variants. 13-14 Nov. 1991, National Institute for Biological Standards and Control, Hertfordshire, UK. Vaccine, 1992. 10(10): 723-5). The MDCK based culture platform appears to be more effective in inducing protection as an inactivated vaccine in animals than egg grown influenza virus vaccines (Wood et al. (1989); Katz, J. M. and R. G. Webster, J. Infect. Dis. 1989; 160(2): 191-8).

Virus strains, propagation and purification. Filamentous influenza viruses A/Udorn/72 (H3N2), spherical influenza strains or the recombinant rIAV A/WSN/M^{UD} (H1N1) will be propagated in MDCK cells as previously described (Roberts, P. C., R. A. Lamb, and R. W. Compans, Virology 1998; 240(1):127-37; Roberts, P. C. and R. W. Compans, Proc Natl Acad Sci USA 1998; 95(10): 5746-51).

The rIAV A/WSN/M^{UD} is a recombinant influenza virus harboring segment 7 derived from IAV A/Udorn/72 in an A/WSN background, which confers the ability to produce filamentous virus particles. The rationale for using filamentous strains of influenza virus is that they provide an optimal platform for incorporation of viral proteins and they allow for easy confirmation of incorporation of our chemokine-fusion proteins. While the A/Udorn virus would not be applicable for use as an avian influenza virus, its utility derives from its low pathogenicity which enables efficacy studies employing only moderate biosafety levels (BSL2). This approach can be used with any current vaccine strain of influenza virus, including vaccines designed for human use. By using dual infection protocols, it has been possible to generate filamentous particles harboring H1 and H3 hemagglutinins within the same particle. Presentation of particles with multiple HA serotypes may induce a more robust heterotypic response than particles presenting only one serotype.

The invention includes a purification protocol that retains particle integrity and is more effective in removing cellular contaminants. Briefly, supernatants harvested from MDCK-infected cells are precleared of cellular debris by low speed centrifugation (800×g, 4° C., 10 min.) Virus is then concentrated by centrifugation through a 14% Optiprep (Axis-Schield) cushion (60 min, 88,000×g, 10° C.), followed by banding over a 14-26% Optiprep gradient (45 min, 75,000×g, 10° C.). Banded virus is collected and concentrated by ultracentrifugation (75,000×g, 45 min, 10° C.) followed by resuspension in PBS. Virus will routinely be subjected to inactivation prior to the final concentration step.

Plasmid-based vectors for cytokine/chemokine expression. In order to incorporate chicken chemokines/cytokines directly into virus particles, these molecules were expressed as fusion constructs linked to the transmembrane and cytoplasmic tail domains of the major viral surface glycoproteins, the HA and NA. It has already been demonstrated that chicken IL-2 can be expressed as a fusion protein linked to the transmembrane and short cytoplasmic tail domain of the viral neuraminidase, NA˜chIL2 in MDCK cells (see Example 1). Importantly, chIL2 bioactivity was demonstrated at the surface of the stably-transfected MDCK cells (clone 15). In addition, it has already been demonstrated that NA˜chIL2 protein was incorporated into filamentous influenza virus particles budding from the surface of the infected cells (see FIG. 4, Example 1).

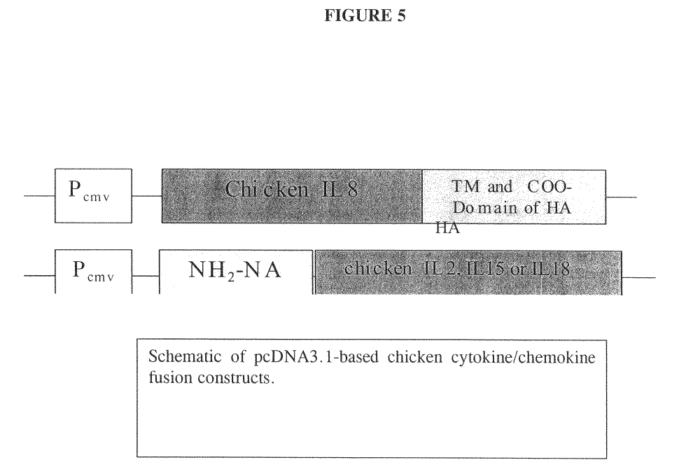

Using standard PCR techniques and specific oligonucleotides, fusion constructs are made consisting of coding regions for the N-terminal 6 amino acid (a.a.) cytoplasmic tail domain, the 29 a.a.-transmembrane domain, and the first 17 amino acids of the stalk domain of the N1 neuraminidase (A/WSN) are fused to the coding regions of chicken IL15 and IL18 minus their N-terminal signal sequence. In the case of chicken IL2, IL15 and IL18, the bioactive COOH domains are extended extracellularly. For IL8, its mature secreted fragment is fused to the HA transmembrane and cytoplasmic tail domains, thus allowing the NH2-end of IL8 to be exposed. A schematic of the constructs is provided in FIG. 5. Since antibodies are not available for chicken IL15, IL18 and IL8, a histidine/flag tag is inserted into each of the constructs at the COOH end of the stalk to facilitate identification of the transcripts on MDCK cells and viral particles.

### Example 3

### Construction of Stable Cell Lines Constitutively Expressing Murine Specific Cytokines Fused to Viral Envelope Proteins

This Example describes the construction of stable cell lines that constitutively express murine specific cytokines fused to viral envelope proteins of influenza. Mouse-specific immunomodulatory molecules can be incorporated directly into virus particles by fusing them to the transmembrane and cytoplasmic tail domains of the viral hemagglutinin and neuraminidase glycoproteins. These immunomodulatory molecules retain bioactivity and induce more robust and effective immune responses in mice and thus serve as a mammalian model for human vaccines bearing human immunomodulators.

A. Construction of Stable Cell Lines Constitutively Expressing Mouse IL2

Constitutive Expression of NA˜mIL2 in MDCK cells. An expression plasmid was generated based on the commercially available pcDNA3.1 in which the coding region of the mature form of the mouse IL2 is fused to the N-terminus coding region of the A/WSN neuraminidase gene. Thus, the COOH end of the mouse IL2 molecule was exposed extracellularly. This embodiment is the same as described for the construction of the chicken IL-2 fusion construct (see Example 1), except for the use of the mature mouse IL2 coding region.

Using PCR, a NA˜mIL2 construct was produced and inserted into the BamHI/EcoRI site present in the multiple cloning site of pcDNA3.1. This construct codes for a protein containing the N-terminal 6 amino acid cytoplasmic tail domain, the 29 amino acid transmembrane domain and the first 16 amino acids of the stalk domain of the N1 protein fused to the mature mouse IL2 protein (minus the signal peptide). An additional linker amino acid (glycine) was inserted between the NA and mIL2 coding region to maintain inframe coding (the glycine was inserted after amino acid residue 51 of the NAmIL2 protein sequence, set forth as SEQ ID NO:20). Initially, the NA and mIL2 (Genbank Accession No. NM_{—}008366; SEQ ID NO:14) DNA sequences were amplified and ligated to each other to produce a NAmIL2 fusion construct.

The primers used for amplification were as follows:

| | |
|---|---|
| 1) Forward primer NA: | |
| 5′-ACTGAATTCTGCCATGAATCCAAACCAGA-3′. | (SEQ ID NO:15) |
| 2) Reverse primer NA: | |
| 5′-TCCGGATCCATTGAGGGCTTGTTGA-3′. | (SEQ ID NO:16) |

This primer is 3′- of the Sty1 site in NA, thus following amplification, a restriction digest with Sty1 results in the appropriate “sticky end” for ligation with the mouse IL2.

3) Forward Primer mIL2:

(SEQ ID NO:17)

5′-TTACCAAGGCGCACCCACTTCAAGCTCCACTTCAAGCTC
(StyI site).
4) Reverse Primer mIL2:

(SEQ ID NO:18)

5′-GAAGAATTCATTCATTGAGGGCTTGTTGAGATGATGCTTTGA-3′
(EcoRI site).

For the above primers, restriction endonuclease cut sites are indicated in italics.

Primers 3 and 4 were used to amplify the mature IL2 protein from plasmid pBR337˜mIL2. Primer 3 contains a Sty1 restriction site and primer 4 contains an EcoRI restriction site. Primers 1 and 2 were used to amplify the N-terminus coding region of the neuraminidase from plasmid pPol-NA (WSN), containing the entire coding region of the NA gene derived from influenza A/WSN/34. Following isolation and purification of both fragments of DNA, they were both cut with the restriction enzyme Sty1, and ligated together to make the construct NAmIL2. The NAmIL2 construct was cloned into the expression vector pcDNA3.1 using BamHI and EcoRI restriction sites.

The NAmIL2 nucleotide sequence and respective protein sequence are set forth as SEQ ID NO:19 and SEQ ID NO:20, respectively. The 5′-BamHI endonuclease restriction site is contained within SEQ ID NO:19 at nucleotides 5-10 and the 3′-EcoRI endonuclease restriction site is contained within SEQ ID NO:19 at nucleotides 620-625.

B. Construction of Stable Cell Lines Constitutively Expressing Mouse GM-CSF.

The mouse GM-CSF coding region (Genbank Accession No. X03019; SEQ ID NO:21) was fused to the carboxy-terminus of the HA coding region. Initially, the carboxy-terminus, coding region of the HA derived from influenza virus A/WSN/34 strain, was amplified by PCR using primers 7 (HA1599-F) (CCGGATCCTCAATGGGGGTGTATC) (SEQ ID NO:24), 8 (HA1513-F) (CCGGATCCAATGGGACTTATGATTATCC) (SEQ ID NO:25), and 9 (HA1730-R) (CCGAATTCTCAGATGCATATTCTGCACTGC) (SEQ ID NO:26), and inserted into pcDNA3.1 using restriction sites BamHI and EcoRI. This construct has the advantage that any fusion construct can be inserted into the HindIII and BamHI site, resulting in an inframe fusion construct with the HA-coding region at the carboxy or 3′ end. Two HA coding regions were constructed in this fashion; the HA1513 codes for nucleotides 1521 to 1730 and the HA1599 codes for nucleotides 1599 to 1730 using primers 7 and 9 and 8 and 9, respectively. They differ only in the length of the extracellular stalk coding region (HA1513 encodes for an additional 26 amino acids). In the example of the mouse GM-CSF˜HA fusion construct, the mouse GM-CSF coding region was amplified by PCR using primers 5 (ACTAAGCTTGGAGGATGTGGCTGCAGA) (SEQ ID NO:22) and 6 (GGGGATCCTTTTTGGACTGGTTTTTTGC) (SEQ ID NO:23). For each of the above primers, restriction endonuclease cut sites are indicated in italics.

The DNA fragment was isolated, treated with restriction endonucleases EcoRI and BamHI and inserted by standard ligation protocols into the HindIII/BamHI site of pcDNA3.1/HA1513 or HA1599. Hereinafter, these constructs are referred to as pcDNA3.1/mGM-CSF˜HA¹⁵¹³ or ˜HA¹⁵⁹⁹.

The nucleotide sequence of the HA1513 Construct inserted into pcDNA3.1 via EcoRI and BamHI restriction sites, hereinafter referred to as pcDNA3.1˜HA1513, is set forth as SEQ ID NO:27. The BamHI endonuclease restriction site is contained within SEQ ID NO:27 at nucleotides 1-6 and the EcoRI endonuclease restriction site is contained within SEQ ID NO:27 at nucleotides 217-222.

The nucleotide sequence of the HA1599 Construct inserted into pcDNA3.1 via HindIII and BamHI restriction sites, hereafter referred to as pcDNA3.1˜HA1599, is set forth as SEQ ID NO:28. The BamHI endonuclease restriction site is contained within SEQ ID NO:28 at nucleotides 1-6 and the EcoRI endonuclease restriction site is contained within SEQ ID NO:28 at nucleotides 138-143.

Both plasmids can be routinely used to make in frame fusion constructs with cytokines, chemokines or costimmulatory molecules fused to the carboxy terminus of the HA coding region. The HA1599 results in a fusion construct just proximal to the transmembrane spanning domain of the HA protein (H1 serotype, derived from influenza A/WSN/34 strain of virus). The sequences of the fusion constructs in pcDNA3.1 are as follows:

The mouse GM-CSF˜HA¹⁵¹³ nucleotide sequence in pcDNA3.1, referred to hereafter as pcDNA3.1/mGM-CSF˜HA¹⁵¹³, is set forth as SEQ ID NO:29.

The protein sequence for mouse GM-CSF HA¹⁵¹³ is set forth as SEQ ID NO:30.

The mouse GM-CSF˜HA¹⁵⁹⁹ nucleotide sequence in pcDNA3.1, referred to hereafter as pcDNA3.1/mGM-CSF˜HA¹⁵⁹⁹, is set forth as SEQ ID NO:31.

The protein sequence for mouse GM-CSF˜HA¹⁵⁹⁹ is set forth as SEQ ID NO:32.

C. Construction of MDCK cell lines that constitutively express mouse NAmIL2 and mouse GM-CSF˜HA¹⁵¹³.

The new plasmid constructs, pcDNA3.1NAmIL2 and pcDNA3.1 mGMCSF˜HA¹⁵¹³ were transfected into MDCK cells (ATCC) using. Lipofectamine 2000 (Invitrogen™) as described by the manufacturer. The transfected cells were then selected for growth in G418 (1.5 mg/ml). Surviving cells were cloned by limiting dilution and screened for cell surface expression of NA˜mIL2 and HA-GM-CSF by standard immunofluorescence staining protocols using monoclonal antibodies specific to mouse IL-2 and mouse GM-CSF.

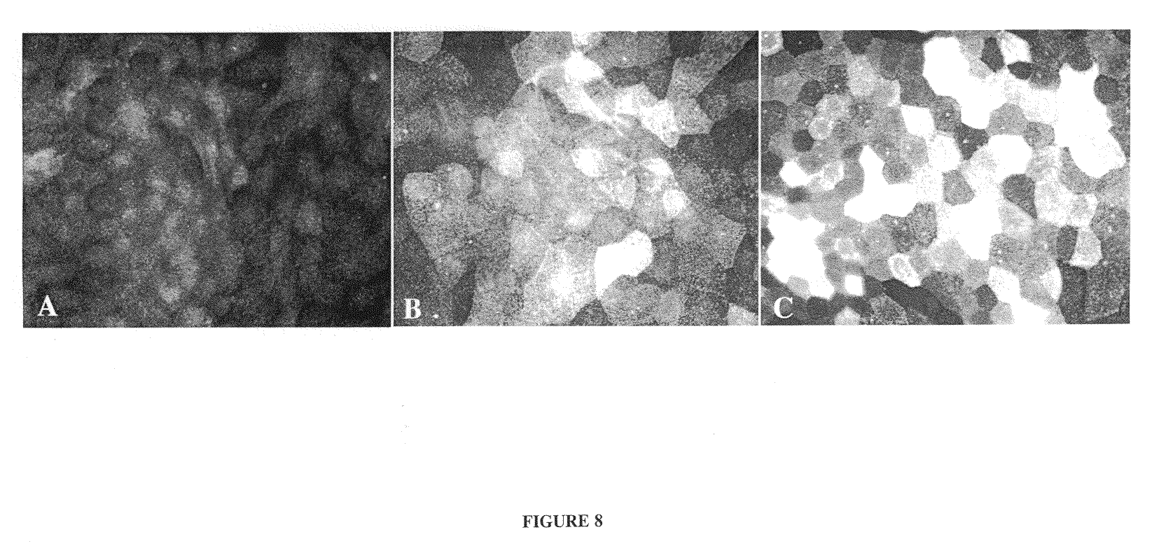

As depicted in FIG. 8, MDCK subclones were isolated, which readily express mouse IL2 or mouse GM-CSF at the cell surface of cells as determined by immunofluorescence microscopy. In order to confirm that the NAmIL2 and mGM-CSF˜HA1513 expressed at the cell surface of MDCK cells were biologically active, an in vitro bioassay was performed.

The mouse IL2 bioassay was based on Current Protocols in Immunology, suppl. 15, pg:6.3.2. Briefly, MDCK cells, transfected with pcDNA3.1-NA-mIL2 or vector control transfected MDCK cells were plated in 96 well flat-bottom plates and incubated until confluent. Then the cells were treated with mitomycin C, 50 ug/ml for 1 to 1.5 hours and washed. Then CTLL2 cells (ATCC™) which had been maintained in RPMI1640 medium supplemented with fetal calf serum (10%), L-glutamine, 2 mM, penicillin/streptomycin, Hepes, 20 mM, pyruvate 2 mM, 2mercaptoethanol (0.1% of 20 uM) and 5 ng/ml rmIL-2 (Biosource™) were collected in active log-phase growth, washed and added to the wells, 5×10³ per well (in RPMI1640 medium supplemented with all of the above, except rmIL2). The plate was incubated for 24 hours at 37° C. ³H-thymidine, 1 μCi was then added and the plate incubated for an additional 24 hours, harvested with an automated harvester and counted for the uptake of thymidine in a liquid scintillation counter. Controls included CTLL-2 cells incubated with dilutions of rmIL-2 (Biosource™).

The mouse GM-CSF bioassay was based on Poloso N J et al. Mol 1 mm. 38: 2001, 803-816 and Current Prot Immunol. Suppl 18, pgs. 6.4.1-8. Briefly, MDCK cells, transfected with pcDNA3.1-HA-mGM-CSF or vector control transfected MDCK cells were plated in 96 well flat-bottom plates and incubated until confluent. Then the cells were treated with mitomycin C, 50 μg/ml for 1 to 1.5 hours and washed.

Mouse bone marrow cells, freshly isolated from the femurs of an adult mouse, washed and resuspended in RPMI1640 supplemented with FCS-10, L-glut-2 mmole, p/s, Hepes, 20 mM, pyruvate 2 mM, 2me-0.1% of 20 uM, were added to the wells (10⁵ cells per well). The plate was incubated for 2 and 1/2 days at 37° C. Then 1 μCi ³H-thymidine was added for the last 18 hours and the plates harvested with an automated harvester and counted for the uptake of thymidine in a liquid scintillation counter. Controls included bone marrow cells incubated with dilutions of rmGM-CSF (Biosource™).

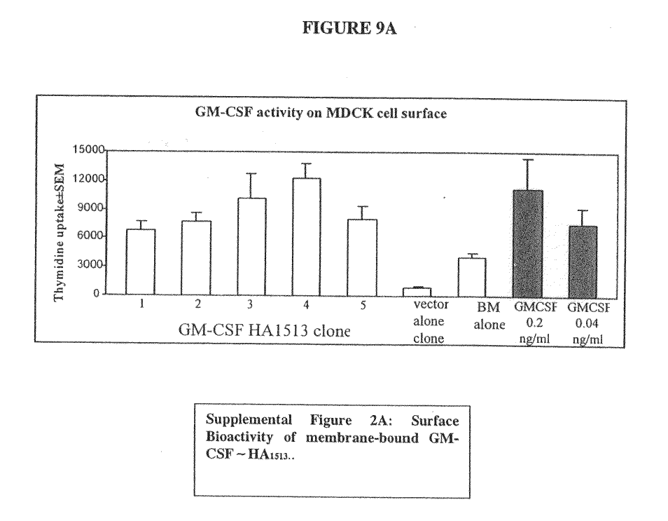

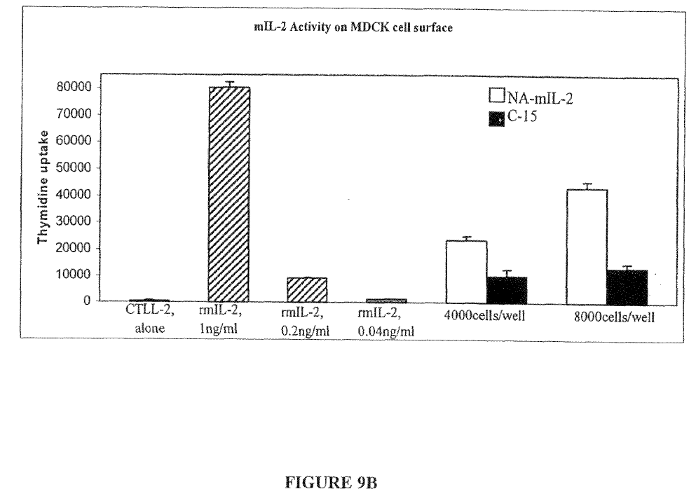

The results (FIGS. 9A and 9B) clearly demonstrate that the surface expression of murine IL2 and GM-CSF in a membrane-bound form fused to influenza virus NA and HA transmembrane and cytoplasmic tail domains is biologically active.

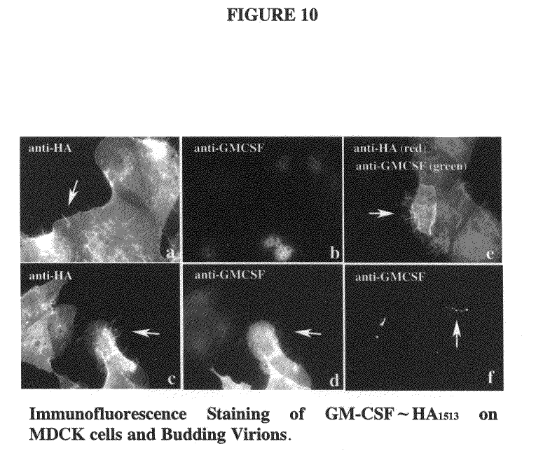

To confirm that virus infection leads to incorporation of membrane-bound mouse cytokines directly into viral particles, immunofluorescence microscopy was used to examine the incorporation of mGM-CSF/HA1513 into budding filamentous influenza virions of influenza A/Udorn/72 infected MDCK/mGM-CSF expressing cells. Positive staining of budding virions (FIGS. 10c,d and e) and released filamentous virions (FIG. 10f) was observed in infected MDCK/mGM-CSF cells but not in MDCK/pcDNA3.1 vector control infected cells (FIGS. 10a and b). This confirms that membrane bound immunomodulators linked to the transmembrane and cytoplasmic tail domains of viral proteins are incorporated into budding virus particles. The same approach as described above in Examples 1 and 2 will be used to 1 generate fusion-constructs of NA˜mIL18 and NA˜mIL15. Mouse IL-8 will be fused to the TM/cytoplasmic tail domain of the hemagglutinin gene similar to the mouse GM-CSF construct, which will tether the COOH-end of the chemokine to the membrane and allow its functional NH₂-domain to be exposed.

The stably transfected MDCK cell cultures will be used which constitutively express mouse immunomodulatory proteins, e.g., chemokines and cytokines anchored to a the transmembrane domains of either the viral hemagglutinin or neuraminidase as platforms for the incorporation of these immunomodulatory proteins, e.g., chemokines and cytokines into newly formed virus particles.

In vivo studies to examine the enhancement of immunogenicity using mouse cytokines and chemokines incorporated into current inactivated, vaccine strains will be carried out.

### Example 4

### Assessment of Inactivation Protocols

In this Example, virus inactivation protocols which will allow for retention of the bioactivity of the cytokines and chemokines are assessed.

Virus Inactivation Protocols. Four different fixation protocols that are currently used for viral inactivation are assessed for retention of the bioactivity of the cytokines and chemokines:

Beta-propiolactone: Virus is treated with 0.015 M beta-propiolactone in PBS pH 7.4, for 15 min at RT. The reaction is stopped by the addition of Nathiosulfate (final conc. 0.04 M) (Budowsky, E. I., A. Smirnov Yu, and S. F. Shenderovich, Vaccine, 1993. 11(3): 343-8).

Heat: Whereas heat-inactivation is generally not used for influenza virus vaccine production, recent results suggest it may induce a more balanced T-cytotoxic response than live infectious virus (Cho, Y., et al., J Virol, 2003. 77(8): 4679-84). The optimal protocol for heat-inactivation of our IVACs-bearing immunomodulators is determined as described. Briefly, virus is subjected to incubation at different temperatures and for different time periods beginning with 56° C. and 10 minutes. Incremental increases of 5° C. and 10 min are assessed. Following each treatment, residual infectivity, hemagglutination and bioactivity is determined.

Formalin: Virus (1 mg/ml) is treated for 5 days at 4° C. with 0.025% formalin (Lu, X., et al., J. Virol. 2001; 75(10):4896-901).

UV. Virus is exposed at 6 inches with 1500 μW seconds/cm² UV for 6 min, followed by a 30 min incubation at 37° C. pH 5.0 (Moran, T. M., et al., J. Infect. Dis. 1999; 180(3):579-85).

Following each inactivation protocol, vaccines are tested for complete loss of infectivity by titration of the vaccine in cell culture (MDCK cells) and hens eggs. Here, the lack of cytopathic effect and viral protein expression in cell culture will confirm effective inactivation. The inability of the vaccine to induce hemagglutinating activity in the allantoic fluid of hens eggs will also be confirmed. Initially, the effects of each inactivation treatment will be determined on MDCK cells transfected with cytokines. Then the treated cells will be assayed for cytokine bioactivity (IL2 and IL15 for their induction of proliferation by T cell blasts, IL-18 for its induction of gamma interferon by splenocytes, and IL-8 for its degranulation of neutrophils). UV radiation and P propiolactone treatments both inactivate nucleic acid (β propiolactone is an alkylating agent) and have been widely used for the inactivation of experimental vaccines. It is, therefore, likely that a dose of each will be found that preserves cytokine bioactivity and, simultaneously, inactivates virus. Formalin, which is widely used for the inactivation of commercial vaccines, will also be tested. Formaldehyde cross-links protein, which might reduce cytokine bioactivity. However, Horwitz et al. (1993) showed that some of the biologic properties of IL2 were preserved after glutaraldehyde fixation to a solid matrix (Horwitz, J. I., et al., Mol. Immunol. 1993; 30(11):1041-8). Heat inactivation, which has the advantage of simplicity and safety, (formalin, β propiolactone and UV radiation must be handled with some precautions) is known to kill influenza virus, but the heat sensitivity of each cytokine is unknown and will be determined empirically. Since all of the cytokines and chemokines being tested have 2 or more intrachain disulfide bonds, they will be at least moderately resistant to heat.

### Example 5

### Standardization of Avian Influenza Vaccines (AIVACs) Bearing Immunomodulators

Since standardization of vaccines would facilitate efficacy comparisons, AIVACs that retain immunomodulatory activity will be standardized based on HA content essentially as described by Wood et al. Avian Dis. 1985; 29(3):867-72), using a single radial immunodiffusion assay. In addition, the vaccines will be standardized based on immunomodulatory activity, expressed in terms of immunomodulatory units, IMU (see below for individual cytokine bioassays). Thus, each vaccine dose will be expressed as IMU per μg HA. Therefore, even in the event hemagglutinating activity is severely reduced by fixation protocols, AIVACs will be standardized based on antigenic content and avian immunomodulatory units.

### Example 6

### Assessment of Bioactivity of Chicken Immunostimulatory Proteins

Bioassay for chicken IL-2, IL-15 and IL-4. The bioassay for chicken IL-2 is described in Sundick, R. S, and C. Gill-Dixon, J. Immunol. 1997; 159(2):720-5 and Kolodsick, J. E., et al., Cytokine 2001; (6):317-24). Briefly, chicken spleen cells are activated with Concanavalin A in Iscoves medium for 24 hrs and then supplemented with 2% heat-inactivated chicken serum at 40° C. for an additional 2 to 3 days. The T cell blasts are purified on a Histopaque gradient, counted and added to 96 well plates (2-4×10⁴ cells per well) with 10 fold dilutions of recombinant IL2, in complete medium. At 18-24 hours 1 uCi of ³H-methyl-thymidine, is added to each well and the cultures are incubated for an additional 6 hours. The plates are harvested in an automated harvester and radioactivity retained by the cells is quantitated on filters in a liquid scintillation counter. IL-2 bioactivity in test samples will be compared to the control dilutions of recombinant chicken IL-2. Bioactivity will be reported in terms of units, where 1 unit is the dilution of sample which induces half-maximal uptake of isotope. This assay will also be used to detect and quantitate IL-15 as reported by Lillehoj, Vet. Immunol. Immunopathol. 2001; 82(3-4):229-44. The bioactivity of cell- and virus bound NA˜chIL15 will be compared with control dilutions of soluble IL15 produced from COS7 cells stably transfected with pcDNA3.1-encoding the full-length chIL15. Activated T cell lines of mammals respond to mammalian IL-4 (Current Protocols in Immunology, Suppl. 15, page 6.3.2). Therefore, we will also utilize mitogen activated T cells of chickens as indicators of chicken IL-4 bioactivity.

Bioassay for chIL-18. IL-18 will be quantitated and assessed for bioactivity in terms of its ability to stimulate the synthesis of interferon gamma (Gobel, T. W., et al., J. Immunol. 2003; 171(4):1809-15). Briefly, samples of cells and virus to be tested for IL18 will be incubated with chicken spleen cells. Then at 4 hours RNA will be prepared from the spleen cells, reverse transcribed to cDNA, using oligo-dT as a primer. Then primers for chicken interferon gamma will be used in real-time RT-PCR assays to quantitate the amount of mRNA induced. Results from multiple RNA preparations will be equalized by the use of beta actin primers to amplify beta actin. Results from cell- and virus-bound NA˜chIL18 will be compared to control dilutions of soluble IL18 produced from COS7 cells stably transfected with pcDNA3.1 encoding full-length IL-18.

Bioassay for IL-8. IL-8 bioactivity will be quantitated by the degranulation of chicken heterophils (the avian equivalent of neutrophils) to yield β-glucuronidase (Kogut, M. H., L. Rothwell, and P. Kaiser, Mol. Immunol. 2003; 40(9):603-10). Heterophils will be isolated from the blood of newly hatched chickens and incubated in 96-well microwells containing IL-8 transfected MDCK cells (8×10⁵ heterophils per well) for 1 hour at 39° C. Then the plate will be centrifuged and the supernatant collected and assayed for β-glucuronidase, using a substrate that upon cleavage fluoresces upon exposure to a wavelength of 355 nm. Controls will include non-transfected MDCK cells and dilutions of soluble IL-8 produced in COS7 cells stably transfected with pcDNA3.1 encoding full-length IL-8. Influenza particles bearing IL8 will also be tested in this assay.

### Example 7

### Determination of Humoral and Cellular Immunogenicity of Influenza Vaccines Expressing Immunomodulators

The presence of specific immunomodulators on the surface of whole, inactivated influenza virus vaccines stimulate a more robust humoral and Th1/Tcytotoxic immune response against influenza virus than conventional immunomodulatory-IVACs.

It is necessary to characterize the immunogenicity of the AI-vaccines bearing avian immunomodulators. Importantly, this Example illustrates the determination of the extent these cytokines enhance and extend the range and scope of the protective immune response against influenza compared to conventional inactivated vaccines. Chicken cytokines/chemokines presented in context with viral antigens induce a more robust and balanced humoral and Th1/T cytotoxic protective immune response. In this Example, the following questions are addressed: 1) What is the minimal standardized vaccine dose required to induce seroconversion and cellular immunity? 2) To what extent do avian cytokines/chemokines expand and enhance the protective immunogenicity of avian influenza vaccines. 3) To what extent do avian cytokines/chemokines induce a more balanced Th1 cross-protective response?

Optimization of AI-vaccines bearing Immunomodulators in Chicks. Based on the in vitro results to optimize inactivation protocols (formalin, β-propiolactone, UV irradiation and heat treatment) conditions will be identified (in terms of dose, duration of treatment, etc.) for each of the 4 inactivation protocols that will result in viral inactivation and, simultaneously, preservation of cytokine/chemokine bioactivity. In this Example, the minimal dose of vaccine necessary to achieve seroconversion using conventional vaccine protocols is determined. Influenza A/Udorn/72 wildtype-based killed vaccine preparations will be standardized according to HA content as described above, and administered subcutaneously (s.c.) or intranasally (i.n.) to specific pathogen free (SPF) chicks at 3 different dose levels (0.1, 1 and 10 μg HA). The chicks will be purchased from Charles River Spafas as specific pathogen free fertile eggs homozygous for the MHC B locus. Chicks hatched from these eggs will be raised in DLAR facilities at Wayne State University until 2 weeks of age. At this time, each chick will be wing banded and injected with the inactivated Udorn. These chicks will be trial bled from the jugular vein at 4 weeks of age (3 ml blood to yield 1 ml serum) and given a booster injection administered at the same site and dose as the initial application. At 6 weeks each chick will be bled from the jugular (3 ml blood) and sacrificed by CO₂ asphyxiation. These sera will be tested for antibodies to HA and NA of Udorn by microneutralization and HA and NA standard inhibition assays. Results from these studies will determine the optimal (and suboptimal) dose of inactivated Udorn and route that will stimulate antibody responses.

Inactivated A/Udorn-based AIVACs bearing each of the 4 cytokines (IL2, IL15, IL18 and IL8) will be standardized based on immunomodulatory units and μg HA as outlined above. Vaccination will be performed either s.c. or i.n. using the minimal dose necessary to achieve seroconversion and T cytotoxic activity as determined above. Use of a minimal dose will increase our sensitivity to detect cytokine enhancement. This will specifically determine the extent that chicken immunomodulators enhance humoral and cellular immunity. Table 1 illustrates the vaccination schedule:

TABLE 1
Vaccination Schedule

n

| Groups | Inactivation Protocol | s.c. | i.n. |
|---|---|---|---|
| AIVAC~chIL2 | UV | 10 each/ | 10 each/ |
| | HEAT | 40 total | 40 total |
| | FORMALIN | | |
| | B-PROPIOLACTONE | | |
| AIVAC~chIL15 | UV | 10 each/ | 10 each/ |
| | HEAT | 40 total | 40 total |
| | FORMALIN | | |
| | B-PROPIOLACTONE | | |
| AIVAC~chIL18 | UV | 10 each/ | 10 each/ |
| | HEAT | 40 total | 40 total |
| | FORMALIN | | |
| | B-PROPIOLACTONE | | |
| AIVAC~chIL8 | UV | 10 each/ | 10 each/ |
| | HEAT | 40 total | 40 total |
| | FORMALIN | | |
| | B-PROPIOLACTONE | | |
| AIVAC~control | UV | 10 each/ | 10 each/ |
| | HEAT | 40 total | 40 total |
| | FORMALIN | | |
| | B-PROPIOLACTONE | | |

Following collection of preimmune sera at 2 weeks of age, Spafas chicks will be inoculated s.c. or i.n. with a standardized, minimal dose of AI-vaccines bearing immunomodulators. Blood will be collected 7 and 14 days post-immunization (p.i). At day 14 p.i., chicks will be bled from the jugular, euthanized and spleens will be harvested. The final bleed typically yields 10 ml blood which will be used to obtain 1 ml serum and 7 ml of peripheral blood lymphocytes, PBLs. The sera will be tested as described above for determination of antibody titers to HA and NA using agar gel precipitation assay, HI-test, NI-test and microneutralization assays. The lymphocytes isolated from the blood and spleen will be used in proliferation assays and the spleen cells will also be used in T cytotoxic assays.

Lymphocyte Proliferation Assay. The proliferation assays will be performed essentially as described by Hu et al. (2001) in our laboratory with minor modifications (Hu, W., et al., eds. Current Progress on Avian Immunology Research, ed. K. A. Schat. 2001, American Association Avian Pathologists: Kennett Square. 269-274). Briefly, lymphocytes will be isolated from blood by centrifugation at 400×g, depleted of adherent cells, and incubated in 96-well plates in the presence or absence of specific antigen (5×10⁵ cells with 3 μg antigen) in 200 μl of Iscoves' medium supplemented with 2 mg/ml BSA and 2% chicken serum. The plates will be incubated at 40° C. in 5% CO₂ for 5 days, the last 15 hours with 1 μCi ³H thymidine and 10⁻⁶M fluorodeoxyuridine. Antigen stimulation will be reported as the stimulation index of cell proliferation in the presence of antigen/cell proliferation without antigen. Spleen cells will be teased into single cell suspensions, depleted of adherent cells by a half-hour incubation and then treated as described for blood lymphocytes. This assay will primarily detect the presence of T helper cells primed in vitro with antigen.

T cytotoxic Assay. The T cytotoxic assay will be performed as described by Seo & Webster (J. Virol. 2001; 75(6):2516-25). Briefly, lung epithelial cells derived from MHC matched chicks will be passaged 10 X and used as targets in cytotoxic assays. The lung cells will be infected either with influenza A/Udorn/72 wildtype virus or a vaccinia vector encoding influenza A/Udorn HA or NA. Targets cells (10⁴ cells) will then be incubated with splenocytes isolated from chickens vaccinated 2 weeks earlier in round-bottom 96-well plates at effector to target cell ratios between 25 and 150. The cells will be spun down, incubated 4 hours, spun again and the supernatants harvested to quantitate the release of LDH, using a nonisotopic cytotoxic assay (Promega, Madison, Wis.). Controls will include lung cells, not infected (as targets), and spleen cells from sham vaccinated chickens (as effector cells). Since T cell cytotoxicity provides significant protection against influenza, these assays are expected to provide critical information about the efficacy of cytokine-bearing influenza particles.

Data collection and statistical analyses. The size of the vaccine cohorts was chosen because these numbers are sufficient to achieve statistically significant differences. The shape of the data distribution will be inspected first by statistical analysis to ensure adherence to the assumptions of the statistical models and to check for the possible existence of outlying observations. In the event of asymmetry, log transformations of the data will be made. In the event of possible outlying observations, the data points will be verified and if found to be valid, analyses will be performed with and without the possible influential points. Statistical analysis of differences will be performed using one-way analysis to compare single factors such as seroconversion. Factorial analysis of variance will be used to assess multiple factors and the association of AIVACs dose and immunogenicity (humoral vs. cellular) to determine the importance of cytokines in directing specific immune responses.

### Example 8

### Analysis of the Efficacy of Cytokines on the Surface of Inactivated A/Udorn Vaccine in Enhancing Antibody Responses

This Example describes the in vivo production of anti-viral antibodies by chicks vaccinated with the novel vaccines of the invention as compared to anti-viral antibodies produced by chicks vaccinated with conventional vaccine.

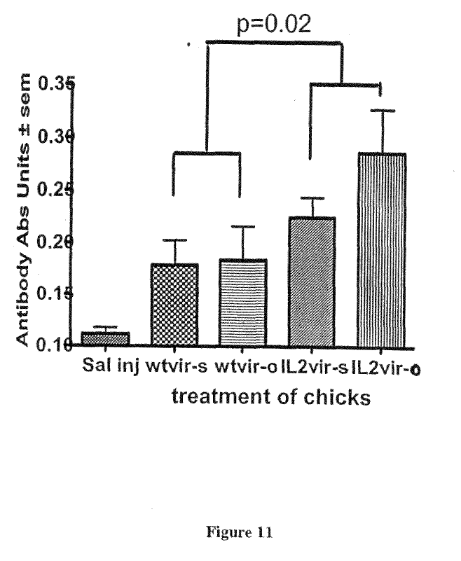

Young chicks (7-9 chicks per group) were vaccinated subcutaneously with conventional avian influenza vaccine (AIVAC) or a novel chIL2 bearing vaccine of the invention (AIVAC˜chIL2). Briefly, the vaccine was UV-inactivated and injected subcutaneously in either PBS or in an oil emulsion formulation (as currently used for poultry vaccines). Chicks were boosted at 21 days and final bleeds were performed at day 14 post-boost. Antibody was evaluated by ELISA using whole virus to coat the wells of a 96 well plate and is expressed in relative optical units (FIG. 11). Sera were diluted 1:100 for testing. Sera was also tested in a hemaglutination-inhibition assay.

As shown in FIG. 11, chicks inoculated with AIVAC-chIL2 (chIL2) in PBS or oil had more antibody than chicks injected with conventional AIVAC (p=0.02). A comparison of the same sera by HI indicated that 7 of 7 chicks injected with IL-2-AIVAC in saline had titers of 20 or more, but only 3 of 7 chicks injected with conventional AIVAC in saline had titers of 20 (Chi square value p=0.018). These results provide important information for further studies concerning dosage, timing, oil emulsion usage and numbers of chickens per group.

### Example 9

### Production of Virus-Like Particles Incorporating Immunomodulatory Molecules

To produce virus-like particles which incorporate membrane-bound immunomodulatory molecules, cell lines (e.g., 293T cells or H9 cells) will be permanently transfected with the genes encoding gag or gag-pol together with the env gene and a fusion-gene encoding a membrane-bound immunomodulatory protein (e.g., human IL-2 or GM-CSF) linked to the transmembrane domain of gp41. During the budding process, virus-like particles will incorporate the cell surface expressed, viral-specific glycoproteins and immunomodulatory fusion proteins as they are released from the cell. The process by which enveloped viruses bud from cells is described in detail (Field's Virology, Fourth Edition, volumes 1 and 2 ed. Knipe and Howley, 2001 (pp 171-197).

### Example 10

### Additional Constructs of Chicken Specific Cytokines and Co-Stimulatory Proteins Fused to Viral HA or NA

A. Construction of Stable Cell Lines Constitutively Expressing Chicken GM-CSF/HA Construct

A full length chicken GM-CSf (Genbank #NM001007078; SEQ ID NO: 33) was synthesized using PCR and primers according to the method of Dillon and Rosen (1999) (Dillon, P. J. and Rosen, C. A., Biotechniques 1999; 9:298-300). The forward primer encoded a HindIII site and the reverse primer encoded the C terminal end of GM-CSF omitting the stop codon and including a BamHI site. This construct was inserted into the pcDNA3.1/HA1513 construct previously described in Example 3(B).

The construct was transfected into MDCK cells. The permanently transfected cells were selected with geneticin, cloned and test for GM-CSK bioactivity as described in Example 3(C), except chicken bone marrow was used as the indicator cells and the media used was Iscoves' medium supplemented with 5% fetal calf serum, 2% autologous chicken serum, 2 mM glutamine, 1 mM pyruvate, penicillin and streptomycin. Cultures were incubated for 3 days at 5% CO₂ at 40° C. In the last 18 hours, 1 uCi of 3H-thymidine was added. The MDCK subclones that were tested for GM-CSF bioactivity were found to have bioactivity.

Influenza virus was then grown on these sublines as well as wild-type MDCK cells, harvested, purified, inactivated with β-propiolactone and tested for GM-CSF bioactivity. The virus was added to 96 plate wells, in triplicate at 3, 10 and 13 micrograms per well with 3×10⁵ chicken bone marrow cells and incubated for 3 days at 40° C. 1 microcurie of 3H-thymidine was added for the last 18 hours. The plate was harvested and counted in a liquid scintillation counter. Controls included bone marrow cells alone and bone marrow cells with Soluble recombinant chicken GM-CSF.

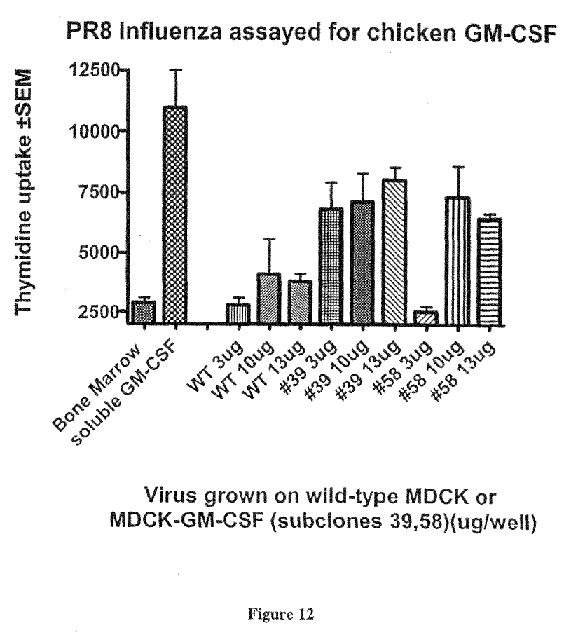

As shown by the results in FIG. 12, only the virus grown on the MDCK cells containing the chGM-CSF/HA construct exhibited significant GM-CSF bioactivity.

B. Construction of Chicken C3d Constructs

The previous examples illustrated expression plasmid generated by inserting chicken cytokines into viral HA or NA. This example illustrates the construction of an expression plasmid generated by inserting a chicken co-stimulatory protein to the HA encoding sequence of HA1513.

Immunomodulatory proteins such as complement component C3d, a B cell stimulatory molecule, lack signal sequences. Thus, the chicken C3d is inserted into the pcDNA3.1IL4ss/HA1513 construct at the BamHI site. This construct contains the signal sequence of chicken IL-4. The coding region of the chicken C3d is amplified using PCR and the following primers:

| | |
|---|---|
| cC3d-F: | |
| ACCAAAGTCAGCATTCAAAGGCACCC | (SEQ ID NO: 34) |
| cC3d-R: | |
| GCGGTAGGTGATGGCGTTGGCG | (SEQ ID NO: 35) |

### Example 11

### Additional Constricts of Murine Specific Cytokines and Co-stimulatory Proteins Fused to Viral HA or NA

This Example illustrates the construction of additional constructs comprising cytokines and co-stimulatory proteins from mice fused to viral HA or NA.

A. Construction of Stable Cell Lines Constitutively Expressing Mouse IL2/HA Construct

An expression plasmid generated by inserting mouse IL2 into the NA has been previously described (Example 3). In this example, an expression plasmid is generated that results in the membrane-bound form of IL2 fused to the HA encoding region of HA1513.

The mouse IL2 coding sequence including its signal sequence was inserted into the KpnI/BamHI site of pcDNA3.1/HA1513 (previously described in Example 3(B)). The mouse IL2 coding region was amplified by PCR using the following primers:

mIL-2 HA KpnI-F

(SEQ ID NO: 36)

(CCGGTACCAGCATGCAGCTCGCATCCTGTGTC);
mIL-2 HA BamHI-R

(SEQ ID NO: 37)

(GGGGATCCTTGAGGGCTTGTTGAGATGA).

For each of the above primers, the restriction endonuclease cut sites are indicated in italics.

The new IL2/HA fusion construct was transfected into MDCK cells as previously described in Example 3(C).

In order to confirm that the mIL2/HA fusion constructs expressed at the surface of the MDCK cells were biologically active, an in vitro bioassay and immunofluorescence were performed as previously described in Example 3(C). Both tests confirmed that the MDCK cells expressed mouse IL2.

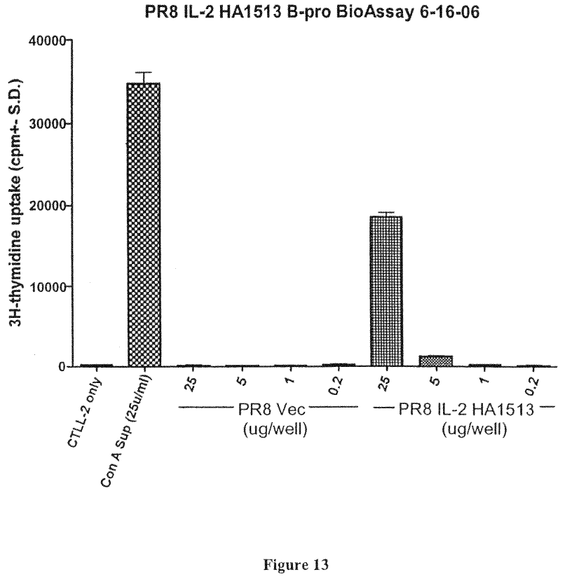

A further bioassay was performed on the MDCK cells containing the mIL2/HA fusion construct also confirmed that the cells were expressing mouse IL2. Influenza A virus A/PR/8 was harvested from the mIL2/HA1513 expressing MDCK cells, purified, and inactivated with β-propiolactone as previously described in Example 4. The virus was then tested for retention of IL2 bioactivity using the CTTL2 indicator cell lines as previously described in Example 3(C). As shown by FIG. 13, significant bioactivity was observed only on virus harboring membrane-bound mIL2/HA.

B. Construction of Stable Cell Lines Constitutively Expressing Mouse IL4/HA Construct

Expression plasmids generated by inserting mouse IL2 into HA and NA has been previously described (Examples 11(A) and 3, respectively). In this Example, mouse IL4 is fused to the HA encoding region of HA1513.

The mouse IL4 coding sequence including its signal sequence was inserted into the KpnI and BamHI site of pcDNA3.1/HA1513 (previously described in Example 3(B)). The mouse IL4 coding region was amplified by PCR using the following primers:

| | |
|---|---|
| mIL-4 HA KpnI-F | |
| (CCGGTACCGCACCATGGGTCTCAACCCCCA); | (SEQ ID NO: 38) |
| mIL-4 HA BamHI-R | |
| (CCGGATCCCGAGTAATCCATTTGCATGATG). | (SEQ ID NO: 39) |

For each of the above primers, the restriction endonuclease cut sites are indicated in italics.

The new IL4/HA fusion construct was transfected into MDCK cells as previously described in Example 3(C).

In order to confirm that the IL4/HA fusion constructs expressed at the surface of the MDCK cells were biologically active, an in vitro bioassay was performed as previously described in Example 3(C), except that the IL4 responsive CT.4s cell line was used instead of the IL2 responsive CTLL2 cell line.

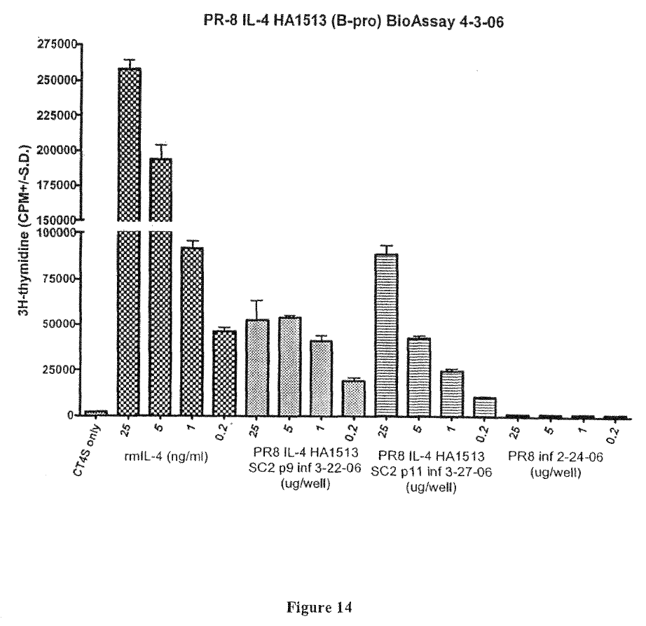

A further bioassay was performed on the MDCK cells containing the mIL4/HA fusion construct also confirmed that the cells were expressing mouse IL4. Influenza A virus A/PR/8 was harvested from the mIL4/HA1513 expressing MDCK cells at two different time points, purified, and inactivated with β-propiolactone as previously described in Example 4. The virus was then tested for retention of IL2 bioactivity using the CT.4s indicator cell lines as previously described in Example 3(C). As shown by FIG. 14, significant bioactivity was observed only on virus harboring membrane-bound mIL4/HA.

C. Construction of Mouse IL4/NA Construct

Mouse IL4 was also inserted into pcDNA3.1/NA25 or pcDNA3.1/NA50 at the BamHI and EcoRI sites. These NA constructs were made from the neuraminidase stalk domains of influenza virus A/WSN/34 strain in order to evaluate whether varying lengths of the neuraminidase stalk domain influence bioactivity of the fused cytokines and/or chemokines. NA25 encodes for the membrane proximal 25 amino acids of the NA stalk and the transmembrane and N-terminal, cytoplasmic tail domains of neuraminidase. The NA 50 construct encodes for an additional 25 amino acids in the stalk domain of the NA. NA 25 and NA50 were inserted into the commercially available pcDNA3.1 plasmid at the BamHI site. These regions of WSN NA were amplified using PCR and the following primers:

| | |
|---|---|
| WSN NA-25: | |
| WSN NA-F: | |
| GGAGATCTAAGATGAATCCAAACCAGAAAATA; | (SEQ ID NO: 40) |
| WSN NA-R: | |
| GGGGATCCAGCAACAACTTTATAGGTAA; | (SEQ ID NO: 41) |
| WSN NA-50: | |
| WSN NA-F: | |
| GGAGATCTAAGATGAATCCAAACCAGAAAATA; | (SEQ ID NO: 42) |
| WSN NA-R: | |
| GGGGATCCGCTGTGTATAGCCCACCCACG. | (SEQ ID NO: 43) |

In each of the above primers, the restriction endonuclease cut sites are indicated in italics.

The coding region of mouse IL4 is then amplified using PCR and the following primers and inserted in the pcDNA3.1/NA25 and pcDNA3.1/NA50 at the BamHI and EcoRI sites.

| | |
|---|---|
| mIL-4 NA BamHI-F: | |
| GGGGATCCCATATCACGGATGCGACA; | (SEQ ID NO: 44) |
| mIL-4 NA EcoRI-R: | |
| CCGAATCCCTACGAGTAATCCATTTGCATGAT. | (SEQ ID NO: 45) |

In each of the above primers, the restriction endonuclease cut sites are indicated in italics.

D. Construction of Mouse IL15 Constructs

In this Example, mouse IL15 was fused to the HA encoding region of HA 1513 and to the two different constructs encompassing the neuraminidase stalk domain.

It has been reported that IL15 is underexpressed due to defects in its signal sequence. Thus, a fusion construct in which the coding region for the signal sequence derived from mouse IL4 was inserted upstream of the pcDNA3.1/HA1513 plasmid. Digestion with the restriction endonuclease BamHI facilitated the incorporation of the sequences bearing either BamHI or BglII restriction sites. The mouse IL-4 signal sequence was amplified by PCR using the following primers:

mIL-4ssHindIII-F:

(SEQ ID NO: 46)

AGCTTCGCCATGGGTCTCAACCCCCAGCTAGTTGTCATCCTGCTCTCTTT
CTCGAATGTACCAGGAGCCATATCG;
mIL-4ss BamHI-R:

(SEQ ID NO: 47)

GATCCGATATGGCTCCTGGTACATTCGAGAAAGAAGAGCAGGATGACAAC
TAGCTGGGGGTTGAGACCCATGGCGA.

In each of the above primers, the restriction endonuclease cut sites are indicated in italics.

Thus, the fusion constructs contain an IL4 signal sequence followed by the coding region of the insert, in this case mouse IL15, tethered inframe to the transmembrane and cytoplasmic tail domains of HA encoded by the HA1513 construct previously described in Example 3(B). This construct was termed pcDNA3.1IL4ss/HA1513.

The coding region of mature IL15 was inserted into the pcDNA3.1IL4ss/HA1513 at the BamHI site. The mouse IL15 coding region was amplified by PCR using the following primers:

| | |
|---|---|
| mIL-15BamHI-F: | |
| CCGGATCCAACTGGATAGATGTAAGATATGACC; | (SEQ ID NO: 48) |
| mIL-15BglI-R: | |
| CCAGATCTGGACGTGTTGATGAACATTT. | (SEQ ID NO: 49) |

In each of the above primers, the restriction endonuclease cut sites are indicated in italics.

Mouse IL-15 was also fused to NA. The coding region of mouse IL15 (without its signal sequence) was amplified using PCR and the following primers and inserted in the pcDNA3.1/NA25 and pcDNA3.1/NA50 at the BamHI and EcoRI sites.

mIL-15 NA BamHI-F:

(SEQ ID NO: 50)

TTAGGATCCAACTGGATAGATGTAAGATATGACCT;
mIL-15 NA EcoRI-R:

(SEQ ID NO: 51)

GAAGAATTCTCATCAGGACGTGTTGATGA.

In each of the above primers, the restriction endonuclease cut sites are indicated in italics.

E. Construction of Mouse C3d Constructs

The previous examples illustrated expression plasmid generated by inserting mouse cytokines into viral HA or NA. This example illustrates that construction of an expression plasmid generated by inserting a mouse co-stimulatory protein to the HA encoding sequence of HA1513.

Immunomodulatory proteins such as complement component C3d, a B cell stimulatory molecule, lack signal sequences. Thus, the mouse C3d was inserted into the pcDNA3.1IL4ss/HA1513 construct at the BamHI site. This construct, described previously in Example 1(C), contains the signal sequence of mouse IL-4. The coding region of the mouse C3d was amplified using PCR and the following primers.

| | |
|---|---|
| mC3d BglII-F: | |
| GGGAGATCTACCCCCGCAAGGCTCTGGG; | (SEQ ID NO: 52) |
| mC3d BamHI-R: | |
| GGGGATCCGAAGGACACATCCATGT. | (SEQ ID NO: 53) |

In each of the above primers, the restriction endonuclease cut sites are indicated in italics.

F. Construction of Stable Cell Lines Constitutively Expressing Mouse Flt3-L/HA Constructs

This example illustrates the insertion of another mouse co-stimulatory protein into pcDNA3.1/HA1513 at the KpnI and BamHI sites. The coding region of the mouse mFlt3-L was amplified using PCR and the following primers.

| | |
|---|---|
| mFlt3-L KpnI-F: | |
| CCGGTACCGCACCATGACAGTGCTGGCGCC; | (SEQ ID NO: 54) |
| mFlt3-L BamHI-R: | |
| CCGGATCCGGGATGGGAGGGGAGGGGCACC. | (SEQ ID NO: 55) |

In each of the above primers, the restriction endonuclease cut sites are indicated in italics.

The fusion constructs were transfected into MDCK cells as previously described in Example 3(C) and tested by immunofluorescence microscopy as also described in Example 3(C). The results showed the membrane bound immunomodulator linked to the viral protein.

G. Construction of Stable Cell Lines Constitutively Expressing Mouse 4 CD40L/NA Constructs

This Example illustrates the insertion of murine CD40-L, a co-stimulatory protein into both the pcDNA3.1/NA25 or pcDNA3.1/NA50 (previously described in Example 11(B)) at the BamHI and EcoRI sites. The coding region of the CD40-L was amplified using PCR and the following primers:

sCD40-L BglII-F:

(SEQ ID NO: 56)

CCAGATCTATGCAAAGAGGTGATGAGGAT;
sCD40-L EcoRI-R:

(SEQ ID NO: 57)

GGGAATTCAGAGTTTGAGTAAGCCAAAAGATGAGA.

In each of the above primers, the restriction endonuclease cut sites are indicated in italics.

The fusion constructs were transfected into MDCK cells as previously described in Example 3(C) and tested by immunofluorescence microscopy as also described in Example 3(C). The results showed the membrane bound immunomodulator expressed on the surface of the transfected MDCK cells.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of which are incorporated herein by reference in their entireties for all purposes.

What is claimed:

1. A composition comprising an enveloped virus expressing an envelope-bound, immunomodulatory protein linked to a viral envelope protein, or to a fragment thereof.

2. The composition of claim 1, wherein the virus is inactivated.

3. The composition of claim 1, wherein the viral envelope protein is a glycoprotein.

4. The composition of claim 1, wherein the immunomodulatory protein is linked to multiple serotypes of the viral envelope protein.

5. The composition of claim 1, wherein the immunomodulatory protein is linked to the amino-terminal domain of the viral envelope protein.

6. The composition of claim 5, wherein the amino terminal domain comprises the transmembrane domain and the cytoplasmic domain of a viral envelope protein.

7. The composition of claim 3, wherein the viral envelope protein is selected from the group consisting of neuramimidase (NA) and hemagglutinin (HA).

8. The composition of claim 1, wherein the immunomodulatory protein is a cytokine, or active fragment thereof.

9. The composition of claim 8, wherein the cytokine is a member selected from the group consisting of IL-1, IL-2, IL-4, IL-5, IL-6, IL-10, IL-12, IL-15, IL-18, GM-CSF, and interferon gamma.

10. The composition of claim 8, wherein the immunomodulatory protein is a chemokine, or active fragment thereof.

11. The composition of claim 10, wherein the chemokine is a member selected from the group consisting of IL-8, SDF-1α, MCP1, MCP2, MCP3 and MCP4 or MCP5, RANTES, MIP-5, MIP-3, eotaxin, MIP-1α, MIP-1β, CMDC, TARC, LARC, and SLC.

12. The composition of claim 1, wherein the immunomodulatory protein is a costimulatory molecule, or active fragment thereof.

13. The composition of claim 12, wherein the costimulatory molecule is a member selected from the group consisting of CD80, CD86, ICAM-1, LFA-3, C3d, CD40-L and Flt3L.

14. The composition of claim 1, wherein the immunomodulatory protein is derived from a animal selected from the group consisting of a chicken, duck, goose, turkey, mouse, horse, cow, sheep, pig, monkey, dog, and cat.

15. The composition of claim 1, wherein the immunomodulatory protein is a human immunomodulatory protein.

16. The composition of claim 1, wherein the virus belongs to the family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Bunyaviridae and Baculoviridae.

17. The composition of claim 16, wherein the virus is selected from the group consisting of human and avian influenza viruses, respiratory syncitial virus (RSV), Hepatitis B. Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

18. A method for producing an enveloped virus expressing an envelope-bound, immunomodulatory protein, the method comprising a) transforming a host cell with an expression vector encoding an immunomodulatory protein and a viral envelope protein, or a fragment thereof, and b) infecting the cell with an enveloped virus, thereby producing an enveloped virus expressing an envelope-bound, immunomodulatory protein.

19. The method of claim 18, wherein the host cell is an MDCK cell.

20. The method of claim 18, further comprising inactivating the virus.

21. The method of claim 18, wherein the virus belongs to the family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Bunyaviridae and Baculoviridae.

22. The method of claim 21, wherein the virus is selected from the group consisting of human and avian influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

23. The method of claim 18, wherein the immunomodulatory protein is a cytokine, or active fragment thereof.

24. The method of claim 23, wherein the cytokine is a member selected from the group consisting of IL-1, IL-2, IL-4, IL-5, IL-6, IL-10, IL-12, IL-15, IL-18, GM-CSF, and interferon gamma.

25. The method of claim 23, wherein the immunomodulatory protein is a chemokine, or active fragment thereof.

26. The method of claim 25, wherein the chemokine is a member selected from the group consisting of IL-8, SDF-1α, MCP1, MCP2, MCP3 and MCP4 or MCP5, RANTES, MIP-5, MIP-3, eotaxin, MIP-1α, MIP-1β, CMDC, TARC, LARC, and SLC.

27. The method of claim 18, wherein the immunomodulatory protein is a costimulatory molecule, or active fragment thereof.

28. The method of claim 27, wherein the costimulatory molecule is a member selected from the group consisting of CD80, CD86, ICAM-1, LFA-3, C3d, CD40-L and FIt3L.

29. The method of claim 18, wherein the immunomodulatory protein is derived from a animal selected from the group consisting of a chicken, duck, goose, turkey, mouse, horse, cow, sheep, pig, monkey, dog, and cat.

30. The method of claim 18, wherein the immunomodulatory protein is a human immunomodulatory protein.

31. The method of claim 18, wherein the viral envelope protein is selected from the group consisting of neuraminidase (NA) and hemagglutinin (HA).

32. A method for inducing an immune response in a animal which comprises administering to the animal an effective amount of a composition comprising an inactive virus expressing an envelope-bound immunomodulatory protein, wherein the immune response induced by the animal is more robust as compared to the immune response that could have been induced in an animal by the virus without the envelope-bound immunomodulatory protein.

33. The method of claim 32, wherein the immunomodulatory protein is linked to a viral envelope protein.

34. The method of claim 32, wherein the immune response is a humoral immune response.

35. The method of claim 32, wherein the immune response is cellular immune response.

36. The method of claim 35, wherein the cellular immune response is a cytotoxic T cell and/or T helper cell mediated immune response.

37. The method of claim 32, wherein the virus belongs to the family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Bunyaviridae and Baculoviridae.

38. The method of claim 37, wherein the virus is selected from the group consisting of human and avian influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

39. The method of claim 32, wherein the animal is selected from the group consisting of a chicken, duck, goose, turkey, mouse, horse, cow, sheep, pig, monkey, dog, and cat.

40. The method of claim 32, wherein the animal is a human.

41. The method of claim 32, wherein the immunomodulatory protein is a cytokine, or active fragment thereof.

42. The method of claim 41, wherein the cytokine is a member selected from the group consisting of IL-1, IL-2, IL-4, IL-5, IL-6, IL-10, IL-12, IL-15, IL-18, GM-CSF, and interferon gamma.

43. The method of claim 41, wherein the immunomodulatory protein is a chemokine, or active fragment thereof.

44. The method of claim 43, wherein the chemokine is a member selected from the group consisting of IL-8, SDF-1α, MCP1, MCP2, MCP3 and MCP4 or MCP5, RANTES, MIP-5, MIP-3, eotaxin, MIP-1β, MIP-1β, CMDC, TARC, LARC, and SLC.

45. The method of claim 32, wherein the immunomodulatory protein is a costimulatory molecule, or active fragment thereof.

46. The method of claim 45, wherein the costimulatory molecule is a member selected from the group consisting of CD80, CD86, ICAM-1, LFA-3, C3d, CD40L and FIt3L.

47. The method of claim 32, wherein the immunomodulatory protein is derived from a animal selected from the group consisting of a chicken, duck, goose, turkey, mouse, horse, cow, sheep, pig, monkey, dog, and cat.

48. The method of claim 32, wherein the immunomodulatory protein is a human immunomodulatory protein.

49. A method for treating or preventing a viral infection in a animal comprising administering to the animal an inactive, enveloped virus expressing an envelope-bound immunomodulatory protein.

50. The method of claim 49, wherein the immunomodulatory protein is linked to a viral envelope protein.

51. The method of claim 49, wherein the virus belongs to the family of viruses selected from the group consisting of Orthomyxoviridae, Herpesviridae, Poxyiridae, African Swine Fever-like Viruses, Hepadnaviridae, Coronaviridae, Flaviviridae, Togaviridae, Retroviridae, Filoviridae, Paramyxoviridae, Rhabdovirisae, Arenaviridae, Bunyaviridae and Baculoviridae.

52. The method of claim 51, wherein the virus is selected from the group consisting of human and avian influenza viruses, respiratory syncitial virus (RSV), Hepatitis B, Hepatitis C, human immunodeficiency virus (HIV), human T-cell leukemia virus (HTLV-1/2), lymphocytic choriomeningitis virus (LCMV), avian sarcoma virus, Herpes, varicella-zoster virus (VZV), cytomegalovirus (CMV), Epstein-Barr virus (EBV), ebola and Marburg viruses, Dengue, West Nile virus, Hantavirus, SARS, small pox, Newcastle disease virus (NDV), infectious bronchitis virus (IBV), infectious laryngotracheitis virus (ILTV), and rabies.

53. The method of claim 49, wherein the viral infection is influenza.

54. The method of claim 53, wherein the viral infection is avian influenza.

55. The method of claim 49, wherein the animal is selected from the group consisting of a chicken, duck, goose, turkey, mouse, horse, cow, sheep, pig, monkey, dog, and cat.

56. The method of claim 49, wherein the animal is a human.

57. The method of claim 49, wherein the immunomodulatory protein is a cytokine, or active fragment thereof.

58. The method of claim 57, wherein the cytokine is selected from the group consisting of IL-1, IL-2, IL-4, IL-5, IL-6, IL-10, IL-12, IL-15, IL-18, GM-CSF, and interferon gamma.

59. The method of claim 57, wherein the immunomodulatory protein is a chemokine, or active fragment thereof.

60. The method of claim 59, wherein the chemokine is a member selected from the group consisting of IL-8, SDF-1α, MCP1, MCP2, MCP3 and MCP4 or MCP5, RANTES, MIP-5, MIP-3, eotaxin, MIP-1α, MIP-1β, CMDC, TARC, LARC, and SLC.

61. The method of claim 49, wherein the immunomodulatory protein is a costimulatory molecule, or active fragment thereof.

62. The method of claim 61, wherein the costimulatory molecule is a member selected from the group consisting of CD80, CD86, ICAM-1, LFA-3, C3d, CD40L and Flt3L.

63. The method of claim 49, wherein the immunomodulatory protein is derived from a animal selected from the group consisting of a chicken, duck, goose, turkey, mouse, horse, cow, sheep, pig, monkey, dog, and cat.

64. The method of claim 49, wherein the immunomodulatory protein is a human immunomodulatory protein.

65. A pharmaceutical composition comprising an enveloped virus expressing an envelope-bound immunomodulatory protein linked to a viral envelope protein and a pharmaceutically acceptable carrier.


SEQUENCE LISTING (57 sequences)

SEQ ID NO: 1 - DNA, 1362 bp, Influenza virus
atgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcagcattacctataaagttgttgctgggcaggactcaacttcagtgatattaaccggcaattcatctctttgtcccatccgtgggtgggctatacacagcaaagacaatggcataagaattggttccaaaggagacgtttttgtcataagagagccttttatttcatgttctcacttggaatgcaggaccttttttctgactcaaggcgccttactgaatgacaagcattcaagggggacctttaaggacagaagcccttatagggccttaatgagctgccctgtcggtgaagctccgtccccgtacaattcaaggtttgaatcggttgcttggtcagcaagtgcatgtcatgatggagtgggctggctaacaatcggaatttctggtccagatgatggagcagtggctgtattaaaatacaaccgcataataactgaaaccataaaaagttggaggaagaatatattgagaacacaagagtctgaatgtacctgtgtaaatggttcatgttttaccataatgaccgatggcccaagtgatgggctggcctcgtacaaaattttcaagatcgagaaggggaaggttactaaatcaatagagttgaatgcacctaattctcactacgaggaatgttcctgttaccctgataccggcaaagtgatgtgtgtgtgcagagacaattggcacggttcgaaccgaccatgggtgtccttcgaccaaaacctagattataaaataggatacatctgcagtggggttttcggtgacaacccgcgtcccaaagatggaacaggcagctgtggcccagtgtctgctgatggagcaaacggagtaaagggattttcatataagtatggcaatggtgtttggataggaaggactaaaagtgacagttccagacatgggtttgagatgatttgggatcctaatggatggacagagactgatagtaggttctctatgagacaagatgttgtggcaataactaatcggtcagggtacagcggaagtttcgttcaacatcctgagctaacagggctagactgtatgaggccttgcttctgggttgaattaatcagggggctacctgaggaggacgcaatctggactagtgggagcatcatttctttttgtggtgtgaatagtgatactgtagattggtcttggccagacggtgctgagttgccgttcaccattgacaagtag

SEQ ID NO: 2 - protein, 453 aa, Influenza virus
MNPNQKIITIGSICMVVGIISLILQIGNIISIWISHSIQTGNQNHTGICNQGSITYKVVAGQDSTSVILTGNSSLCPIRGWAIHSKDNGIRIGSKGDVFVIREPFISCSHLECRTFFLTQGALLNDKHSRGTFKDRSPYRALMSCPVGEAPSPYNSRFESVAWSASACHDGVGWLTIGISGPDDGAVAVLKYNRIITETIKSWRKNILRTQESECTCVNGSCFTIMTDGPSDGLASYKIFKIEKGKVTKSIELNAPNSHYEECSCYPDTGKVMCVCRDNWHGSNRPWVSFDQNLDYKIGYICSGVFGDNPRPKDGTGSCGPVSADGANGVKGFSYKYGNGVWIGRTKSDSSRHGFEMIWDPNGWTETDSRFSMRQDVVAITNRSGYSGSFVQHPELTGLDCMRPCFWVELIRGLPEEDAIWTSGSIISFCGVNSDTVDWSWPDGAELPFTIDK

SEQ ID NO: 3 - DNA, 432 bp, Gallus gallus
atgatgtgcaaagtactgatctttggctgtatttcggtagcaacgctaatgactacagcttatggagcatctctatcatcagcaaaaaggaaacctcttcaaacattaataaaggatttagaaatattggaaaatatcaagaacaagattcatctcgagctctacacaccaactgagacccaggagtgcacccagcaaactctgcagtgttacctgggagaagtggttactctgaagaaagaaactgaagatgacactgaaattaaagaagaatttgtaactgctattcaaaatatcgaaaagaacctcaagagtcttacgggtctaaatcacaccggaagtgaatgcaagatctgtgaagctaacaacaagaaaaaatttcctgattttctccatgaactgaccaactttgtgagatatctgcaaaaataa

SEQ ID NO: 4 - protein, 143 aa, Gallus gallus
MMCKVLIFGCISVATLMTTAYGASLSSAKRKPLQTLIKDLEILENIKNKIHLELYTPTETQECTQQTLQCYLGEVVTLKKETEDDTEIKEEFVTAIQNIEKNLKSLTGLNHTGSECKICEANNKKKFPDFLHELTNFVRYLQK

SEQ ID NO: 5 - DNA, 27 bp, artificial
  primer
gactggatccctgccatgaatccaaac

SEQ ID NO: 6 - DNA, 6 bp, artificial
  synthetic sequence
ggatcc

SEQ ID NO: 7 - DNA, 5 bp, artificial
  synthetic sequence
ctgcc

SEQ ID NO: 8 - DNA, 18 bp, artificial
  primer
actgccttggttgcatat

SEQ ID NO: 9 - DNA, 6 bp, artificial
  synthetic sequence
ccttgg

SEQ ID NO: 10 - DNA, 23 bp, artificial
  primer
gcatccaaggcgcatctctatca

SEQ ID NO: 11 - DNA, 6 bp, artificial
  synthetic sequence
ccaagg

SEQ ID NO: 12 - DNA, 21 bp, artificial
  primer
gctagaattcttatttttgca

SEQ ID NO: 13 - DNA, 6 bp, artificial
  synthetic sequence
gaattc

SEQ ID NO: 14 - DNA, 939 bp, Mus musculus
atcacccttgctaatcactcctcacagtgacctcaagtcctgcaggcatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataactatgtacctcctgcttacaacacataaggctctctatttatttaaatatttaactttaatttatttttggatgtattgtttactatcttttgtaactactagtcttcagatgataaatatggatctttaaagattctttttgtaagccccaagggctcaaaaatgttttaaactatttatctgaaattatttattatattgaattgttaaatatcatgtgtaggtagactcattaataaaagtatttagatgattcaaatataaataagctcagatgtctgtcatttttaggacagcacaaagtaagcgctaaaataacttctcagttattcctgtgaactctatgttaatcagtgttttcaagaaataaagctctcctct

SEQ ID NO: 15 - DNA, 29 bp, artificial
  primer
actgaattctgccatgaatccaaaccaga

SEQ ID NO: 16 - DNA, 25 bp, artificial
  primer
tccggatccattgagggcttgttga

SEQ ID NO: 17 - DNA, 39 bp, artificial
  primer
ttaccaaggcgcacccacttcaagctccacttcaagctc

SEQ ID NO: 18 - protein, 42 aa, artificial
  synthetic protein
GAAGAATTCATTCATTGAGGGCTTGTTGAGATGATGCTTTGA

SEQ ID NO: 19 - DNA, 628 bp, artificial
  synthetic construct
gactggatccctgccatgaatccaaaccagaaaataataaccattgggtcaatctgtatggtagtcggaataattagcctaatattgcaaataggaaatataatctcaatatggattagccattcaattcaaaccggaaatcaaaaccatactggaatatgcaaccaaggcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaatgaattccgg

SEQ ID NO: 20 - protein, 201 aa, artificial
  fusion protein
MNPNQKIITIGSICMVVGIISLILQIGNIISIWISHSIQTGNQNHTGICNQGAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 21 - DNA, 781 bp, Mus musculus
actcagagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaatgaggaagcccaggccagctctgaatccagcttctcagactgctgcttttgtgcctgcgtaatgagccaggaactcggaatttctgccttaaagggaccaagagatgtggcacagccacagttggagggcagtatagccctctgaaaacgctgactcagcttggacagcggaagacaaacgagagatattttctactgatagggaccattatatttatttatatatttatatttttaaatatttatttatttatttatttaattttgcaactctatttattgagaatgtcttaccagaataataaattattaaaacttttgtttgt

SEQ ID NO: 22 - DNA, 27 bp, artificial
  primer
actgaattcggaggatgtggctgcaga

SEQ ID NO: 23 - DNA, 28 bp, artificial
  primer
ggggatcctttttggactggttttttgc

SEQ ID NO: 24 - DNA, 24 bp, artificial
  primer
ccggatcctcaatgggggtgtatc

SEQ ID NO: 25 - DNA, 28 bp, artificial
  primer
ccggatccaatgggacttatgattatcc

SEQ ID NO: 26 - DNA, 30 bp, artificial
  primer
ccgaattctcagatgcatattctgcactgc

SEQ ID NO: 27 - DNA, 222 bp, artificial
  construct
ggatccaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctgagaattc

SEQ ID NO: 28 - DNA, 144 bp, artificial
  synthetic construct
ggatcctcaatgggggtgtatcagattctggcgatctactcaactgtcgccagttcactggtgcttttggtctccctgggggcaatcagtttctggatgtgttctaatgggtctttgcagtgcagaatatgcatctgagaattc

SEQ ID NO: 29 - DNA, 652 bp, artificial
  synthetic construct
ccaagcttggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaaggatccaatgggacttatgattatccaaaatattcagaagaatcaaagttgaacagggaaaagatagatggagtgaaattggaatcaatgggggtgtatcagattctggcgatctactcaactgtcgccagttcactggtgcttttggtctccctgggggcaatcagtttctggatgtgttctaatgggtctttgcagtgcagaatatgcatctga

SEQ ID NO: 30 - protein, 212 aa, artificial
  fusion protein
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGSNGTYDYPKYSEESKLNREKIDGVKLESMGVYQILAIYSTVASSLVLLVSLGAISFWMCSNGSLQCRICI

SEQ ID NO: 31 - DNA, 574 bp, artificial
  synthetic construct
ccaagcttggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacattgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccagtccaaaaaggatcctcaatgggggtgtatcagattctggcgatctactcaactgtcgccagttcactggtgcttttggtctccctgggggcaatcagtttctggatgtgttctaatgggtctttgcagtgcagaatatgcatctga

SEQ ID NO: 32 - protein, 186 aa, artificial
  fusion protein
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPVQKGSSMGVYQILAIYSTVASSLVLLVSLGAISFWMCSNGSLQCRICI

SEQ ID NO: 33 - DNA, 435 bp, Gallus gallus
atgctggcccagctcactattctgcttgccctcggggtgctctgcagccctgcgcccaccacaacatactcctgctgctacaaagtgtacaccatcctggaagaaataacgagtcacttggagagcacagcggccacagcaggtctgtcctcggtacccatggacatcagggataaaacctgtctgcgtaacaacctgaaaacattcatagagtccttgaaaacaaatgggacagaggaagaaagcggaatcgtctttcagctgaacagagttcacgagtgtgaacgcctcttctcgaacataactcccaccccgcaggttcctgataaggaatgtagaactgcacaagtatcgagggaaaaattcaaagaggcattaaaaactttctttatttacctctctgatgtgctcccagaggagaaagactgcatctaa

SEQ ID NO: 34 - DNA, 26 bp, artificial
  primer
accaaagtcagcattcaaaggcaccc

SEQ ID NO: 35 - DNA, 22 bp, artificial
  primer
gcggtaggtgatggcgttggcg

SEQ ID NO: 36 - DNA, 32 bp, artificial
  primer
ccggtaccagcatgcagctcgcatcctgtgtc

SEQ ID NO: 37 - DNA, 28 bp, artificial
  primer
ggggatccttgagggcttgttgagatga

SEQ ID NO: 38 - DNA, 30 bp, artificial
  primer
ccggtaccgcaccatgggtctcaaccccca

SEQ ID NO: 39 - DNA, 30 bp, artificial
  primer
ccggatcccgagtaatccatttgcatgatg

SEQ ID NO: 40 - DNA, 32 bp, artificial
  primer
ggagatctaagatgaatccaaaccagaaaata

SEQ ID NO: 41 - DNA, 27 bp, artificial
  primer
gggatccagcaacaactttataggtaa

SEQ ID NO: 42 - DNA, 32 bp, artificial; identical to SEQ ID NO: 40
  primer

SEQ ID NO: 43 - DNA, 29 bp, artificial
  primer
ggggatccgctgtgtatagcccacccacg

SEQ ID NO: 44 - DNA, 26 bp, artificial
  primer
ggggatcccatatcacggatgcgaca

SEQ ID NO: 45 - DNA, 32 bp, artificial
  primer
ccgaatccctacgagtaatccatttgcatgat

SEQ ID NO: 46 - DNA, 75 bp, artificial
  primer
agcttcgccatgggtctcaacccccagctagttgtcatcctgctctctttctcgaatgtaccaggagccatatcg

SEQ ID NO: 47 - DNA, 76 bp, artificial
  primer
gatccgatatggctcctggtacattcgagaaagaagagcaggatgacaactagctgggggttgagacccatggcga

SEQ ID NO: 48 - DNA, 33 bp, artificial
  primer
ccggatccaactggatagatgtaagatatgacc

SEQ ID NO: 49 - DNA, 28 bp, artificial
  primer
ccagatctggacgtgttgatgaacattt

SEQ ID NO: 50 - DNA, 35 bp, artificial
  primer
ttaggatccaactggatagatgtaagatatgacct

SEQ ID NO: 51 - DNA, 29 bp, artificial
  primer
gaagaattctcatcaggacgtgttgatga

SEQ ID NO: 52 - DNA, 28 bp, artificial
  primer
gggagatctacccccgcaaggctctggg

SEQ ID NO: 53 - DNA, 25 bp, artificial
  primer
ggggatccgaaggacacatccatgt

SEQ ID NO: 54 - DNA, 30 bp, artificial
  primer
ccggtaccgcaccatgacagtgctggcgcc

SEQ ID NO: 55 - DNA, 30 bp, artificial
  primer
ccggatccgggatgggaggggaggggcacc

SEQ ID NO: 56 - DNA, 29 bp, artificial
  primer
ccagatctatgcaaagaggtgatgaggat

SEQ ID NO: 57 - DNA, 35 bp, artificial
  primer
gggaattcagagtttgagtaagccaaaagatgaga